(19) United States
(12) Patent Application Publication
(10) Pub. No.: US 2003/0114514 A1
(43) Pub. Date: Jun. 19, 2003

(54) Pharmaceutical compositions containing anti-beta 1 integrin compounds and uses

(76) Inventors: Zhongli Zheng, Lexington, MA (US); Julio H. Cuervo, Arlington, MA (US); KoChung Lin, Lexington, MA (US); Humayun Saleem Ateeq, Weymouth, MA (US)

Correspondence Address: HAROLD H. FOX, FISH & RICHARDSON P.C., 1425 K STREET, NW, 11TH FLOOR, WASHINGTON, DC, 20005-3500, US

(21) Appl. No.: 10/202,740

(22) Filed: Jul. 25, 2002

Related U.S. Application Data
(63) Continuation of application No. PCT/US01/02783, filed on Jan. 26, 2001.
(60) Provisional application No. 60/178,585, filed on Jan. 28, 2000.

(51) Int. Cl. A61K 31/4025; A61K 31/401

(52) U.S. Cl. 514/422; 514/423

(57) ABSTRACT

Organic Anti-Beta 1 integrin compounds useful for inhibiting cell-adhesion.

### RELATED APPLICATIONS

This is a continuation of PCT/US01/02783, filed on Jan. 26, 2001, which claims priority from U.S. provisional application Serial No. 60/178,585 filed on Jan. 28, 2000.

### FIELD OF THE INVENTION

The present invention relates to organic compounds which are useful for blocking the activity of integrin molecules containing the beta1 subunit. More particularly, the invention relates to such organic compounds that inhibit and prevent collagen-based leukocyte adhesion and leukocyte adhesion-mediated pathologies. This invention also relates to compositions containing such compounds and methods of treatment using such compounds.

### BACKGROUND OF THE INVENTION

Many physiological processes require that cells come into close contact with other cells and/or extracellular matrix. Such adhesion events may be required for cell activation, migration, proliferation and differentiation. Cell-cell and cell-matrix interactions are mediated through several families of cell adhesion molecules including the selecting, integrins, and immunoglobulins. Such molecules play an essential role in both normal and pathophysiological processes in a wide variety of tissues. For instance, cellular adhesion and trafficking across the vascular interface plays an essential role in both physiological and pathophysiological processes of acute brain injury. (Garcia et al 1994, Am. J. Pathol. 144:188; Becker et al, 1997 PNAS 94:10873). Leukocyte migration into glomeruli is a typical feature of human glomerulonephritis (GN) and leukocytes are key mediators of kidney damage. Blocking the integrin VLA-4 inhibits acute nephrotoxic nephritis in a rat model of this disease (Tam, F. W. K. et al., Nephrol. Dial. Transplant. 14: 1658-1666 (1999).

Therefore, the targeting of specific and relevant molecules in certain disease conditions without interfering with normal cellular functions is essential for an effective and safe therapeutic agent that inhibits cell-cell and cell-matrix interactions.

The integrin very late antigen (VLA) superfamily is made up of structurally and functionally related glycoproteins consisting of (alpha and beta) heterodimeric, transmembrane receptor molecules found in various combinations on nearly every mammalian cell type. (for reviews see: E. C. Butcher, Cell, 67, 1033 (1991); D. Cox et al., “The Pharmacology of the Integrins.” Medicinal Research Rev. (1994) and V. W. Engleman et al., “Cell Adhesion Integrins as Pharmaceutical Targets.” in Ann. Report in Medicinal Chemistry, Vol. 31, J. A. Bristol, Ed.; Acad. Press, NY, 1996, p. 191). Adhesion molecules of the VLA family presently include VLA-1, -2, -3, -4, -5, -6, -9, -10, -11, -v in which each of the molecules comprise a β chain non-covalently bound to an α chain, (α1, α2, α3, α4, α5, α6, α9, α11, αv), respectively.

Collagen is a fibril-forming protein which is essential for maintaining the integrity of the extracellular matrix found in connective tissues. The major cell surface collagen receptors are the α1β1 (VLA-1) and α2β1 (VLA-2) integrins. Both integrins have been implicated in cell adhesion and migration on collagen (Keely et al. (1995) J. Cell Sci. 108:595-607 and Gotwals et al. (1996) J. Clin. Invest. 97: 2469-2477); in promoting contraction of collagen matrices (Gotwals et al. (1996) J. Clin. Invest. 97: 2469-2477) and in regulating the expression of genes involved in the remodeling of the extracellular matrix (Riikonen et al. (1995) J. Biol. Chem. 270:1-5 and Langholz et al. (1995) J. Cell Biol. 131: 1903-1915).

VLA-1, -2, and -6 neutralizing antibodies or blocking peptides that inhibit the interaction between these respective VLA moieties and their ligands are known. Moreover, in the case of VLA-4 (α4β1) and its ligand VCAM-1, some antibody antagonists have proven efficacious both prophylactically and therapeutically in several animal models of disease, including i) experimental allergic encephalomyelitis, a model of neuronal demyelination resembling multiple sclerosis (for example, see T. Yednock et al., “Prevention of experimental autoimmune encephalomyelitis by antibodies against alpha4beta1 integrin.” Nature, 356, 63 (1993) and E. Keszthelyi et al., “Evidence for a prolonged role of alpha4 integrin throughout active experimental allergic encephalomyelitis.” Neurology, 47, 1053 (1996)); ii) bronchial hyperresponsiveness in sheep and guinea pigs as models for the various phases of asthma (for example, see W. M. Abraham et al., “Alpha 4-Integrins mediate antigen-induced late bronchial responses and prolonged airway hyperresponsiveness in sheep.” J. Clin, Invest, 98, 776 (1993) and A. A. Milne and P. P. Piper, “Role of VLA-4 integrin in leukocyte recruitment and bronchial hyperresponsiveness in the gunea-pig.” Eur. J. Pharmacol., 282, 243 (1995)); ix) tumor metastasis (for examples, see M. Edward, “Integrins and other adhesion molecules involved in melanocytic tumor progression.”, Curr, Opin. Oncol., 7, 185 (1995)).

Several peptidyl antagonists specific to VLA-4 have been described (D. Y. Jackson et al., “Potent α4β1 peptide antagonists as potential anti-inflammatory agents', J. Med. Chem., 40,3359 (1997); U.S. Pat. No. 5,510,332, PCT Publications W097/03094, W097/02289, W096/40781, W096/22966,W096/20216, W096/01644, W096106108, and W095/15973).

Notwithstanding the fact that peptidyl antagonists to α4β1 have been prepared, there remains a need for low molecular weight, specific inhibitors of other integrins besides VLA-4 (e.g., VLA-1, -2, -6 and others). In particular, more than one integrin may be present under certain normal or abnormal physiological conditions, and there is a need to develop low molecular weight, specific inhibitors of any single integrin or plurality of integrins that contain the β1 subunit. Such inhibitors would have improved pharmacokinetic and pharmacodynamic properties such as oral bioavailability and significant duration of action when compared to peptidyl antagonists. Such “pan-β1” antagonist compounds would be useful for the treatment, prevention or suppression of various pathologies mediated by VLA binding and cell adhesion and activation.

### SUMMARY OF THE INVENTION

We have surprisingly discovered that certain potent α4β1 peptide antagonists will also antagonize other members of the family of β1 subunit containing integrins. We have developed methods of using these pan-β1 antagonists that are useful in inhibiting β1 subunit containing integrins and, in so doing, are useful in inhibiting cell adhesion processes including cell activation, migration, proliferation and differentiation.

One aspect of the present invention provides a method for antagonizing the action of a plurality of β1 subunit containing integrins, useful in the treatment of diseases, disorders, conditions or symptoms mediated by cell adhesion in a mammal. The method comprises administering to a cell or other system containing the integrin an effective amount of a pan-β1 antagonist compound of Formula 1:

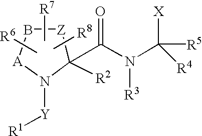

or a pharmaceutically acceptable salt thereof wherein:

R¹ is

1) C1-10 alkyl,

2) C2-10 alkenyl,

3) C2-10 alkynyl,

4) Cy,

5) Cy—C1-10 alkyl,

6) Cy—C2-10 alkenyl,

7) Cy—C2-C10 alkynyl,

wherein alkyl, alkenyl, and alkynyl are optionally substituted with one to four substituents independently selected from R^{a}; and Cy is optionally substituted with one to four substituents independently selected from R_{b};

R² is

1) hydrogen,

2) C1-10 alkyl,

3) C2-10 alkenyl,

4) C2-10 alkynyl,

5) aryl,

6) aryl-C1-10 alkyl,

7) heteroaryl,

8) heteroaryl-C1-10 alkyl,

wherein alkyl, alkenyl, and alkynyl are optionally substituted with one to four substituents independently selected from R^{a} and aryl and heteroaryl are optionally substituted with one to four substituents independently selected from R^{b};

R³ is

1) hydrogen,

2) C1-10 alkyl,

3) Cy, or

4) Cy—C1-10 alkyl,

wherein alkyl is optionally substituted with one to four substituents independently selected from R^{a}; and Cy is optionally substituted with one to four substituents independently selected from R^{b};

R⁴is

1) hydrogen,

2) C1-10 alkyl,

3) C2-10 alkenyl,

4) C2-10 alkynyl,

5) Cy,

6) Cy—C1-10 alkyl,

7) Cy—C2-10 alkenyl,

8) Cy—C2-10 alkynyl,

wherein alkyl, alkenyl and alkynyl are optionally substituted with one to four substituents selected from phenyl and R^{x}, and Cy is optionally substituted with one to four substituents independently selected from R^{y}; or

R³, R⁴ or R³, R⁵ and the atoms to which they are attached together form a mono- or bicyclic ring containing 0-2 additional heteroatoms selected from N, O, and S;

R⁵ is

1) hydrogen,

2) C1-10 alkyl,

3) C2-10 alkenyl,

4) C2-10 alkynyl,

5) aryl,

6) aryl-C1-10 alkyl,

7) heteroaryl,

8) heteroaryl-C1-10 alkyl,

wherein alkyl, alkenyl and alkynyl are optionally substituted with one to four substituents selected from R^{x} and aryl and heteroaryl are optionally substituted with one to four substituents independently selected from R^{y}; or

R⁴, R⁵ and the carbon to which they are attached form a 3-7 membered mono- or bicyclic ring containing 0-2 heteroatoms selected from N, O and S;

R⁶, R⁷, and R⁸ are each independently selected from the group consisting of

1) a group selected from R^{d}, and

2) a group selected from R^{x}; or

two of R⁶, R⁷, and R⁸ and the atom to which both are attached, or two of R⁶, R⁷, and R⁸ and the two adjacent atoms to which they are attached, together form a 5-7 membered saturated or unsaturated monocyclic ring containing zero to three heteroatoms selected from N, O or S,

R¹² is

1) hydrogen,

2) C1-10 alkyl,

3) C2-10 alkenyl,

4) C2-10 alkynyl,

5) Cy,

6) Cy—C1-10 alkyl,

7) Cy—C2-10 alkenyl,

8) Cy—C2-10 alkynyl,

wherein alkyl, alkenyl and alkynyl are optionally substituted with one to four substituents selected from phenyl and R^{x}, and Cy is optionally substituted with one to four substituents independently selected from R^{y};

R¹³ is

1) hydrogen,

2) C1-10 alkyl,

3) C2-10 alkenyl,

4) C2-10 alkynyl,

5) aryl,

6) aryl-C1-10 alkyl,

7) heteroaryl,

8) heteroaryl-C1-10 alkyl,

wherein alkyl, alkenyl and alkynyl are optionally substituted with one to four substituents selected from R^{x} and aryl and heteroaryl are optionally substituted with one to four substituents independently selected from R^{y};

R^{a} is

1) Cy, or

2) a group selected from R^{x};

wherein Cy is optionally subsituted with one to four substituents independently selected from R^{c};

R^{b} is

1) a group selected from R^{a},

2) C1-10 alkyl,

3) C2-10 alkenyl,

4) C2-10 alkynyl,

5) aryl C1-10 alkyl,

6) heteroaryl C1-10 alkyl,

wherein alkyl, alkenyl, alkynyl, aryl, heteroaryl are optionally substituted with a group independently selected from R^{c};

R^{c} is

1) halogen,

2) NO₂,

3) C(O)OR^{f}

4) C1-4 alkyl,

5) C1-4 alkoxy,

6) aryl,

7) aryl C1-4 alkyl,

8) aryloxy,

9) heteroaryl,

10) NR^{f}R^{g},

11) NR^{f}C(O)R^{g},

12 NR^{f}C(O)NR^{f}R^{g},

13) CN;

14) C(O) Cy; or

15) C(O) alkyl

R^{d} and R^{e} are independently selected from hydrogen, C1-10 alkyl, C2-10 alkenyl, C2-10 alkynyl, Cy and Cy C1-10 alkyl, aryl, heteroaryl, aryl-substituted aryl, aryl substituted heteroaryl, heteroaryl-substituted heteroaryl

wherein alkyl, alkenyl, alkynyl, heteroaryl, and Cy is optionally substituted with one to four substituents independently selected from R^{e}; or

R^{d} and R^{e} together with the atoms to which they are attached form a heterocyclic ring of 5 to 7 members containing 0-2 additional heteroatoms independently selected from oxygen, sulfur and nitrogen;

R^{f} and R^{g} are independently selected from hydrogen, C1-10 alkyl, Cy and Cy—C1-10 alkyl wherein Cy is optionally substituted with C1-10 alkyl; or R^{f} and R^{g} together with the carbon to which they are attached form a ring of 5 to 7 members containing 0-2 heteroatoms independently selected from oxygen, sulfur and nitrogen, wherein the nitrogen is optionally substituted with C(O)R^{e}, SO₂R^{e}, or SO₂NR^{d}R^{e}

R^{h} is

1) hydrogen,

2) C1-10 alkyl,

3) C2-10 alkenyl,

4) C2-10 alkynyl,

5) cyano,

6) aryl,

7) aryl C1-10 alkyl,

8) heteroaryl,

9) heteroaryl C1-10 alkyl, or

10) —SO₂Rⁱ;

wherein alkyl, alkenyl, and alkynyl are optionally substituted with one to four substituents independently selected from R^{a}; and aryl and heteroaryl are each optionally substituted with one to four substituents independently selected from R^{b};

Rⁱ is

1) C1-10 alkyl,

2) C2-10 alkenyl,

3) C2-10 alkynyl, or

4) aryl;

wherein alkyl, alkenyl, alkynyl and aryl are each optionally substituted with one to four substituents independently selected from R^{c};

R^{j} is selected from hydrogen, C1-10 alkyl, C2-10 alkenyl, C2-10 alkynyl, Cy and Cy C1-10 alkyl, aryl, heteroaryl, aryl-substituted aryl, aryl substituted heteroaryl, heteroaryl-substituted heteroaryl

wherein alkyl, alkenyl, alkynyl, heteroaryl, and Cy is optionally substituted with one to four substituents independently selected from R^{c}

R^{x} is

1) —OR^{d},

2) —NO₂,

3) halogen,

4) —S(O)ₘR^{d}

5) —SR^{d},

6) —S(O)₂OR^{d},

7) —S(O)ₘNR^{d}R^{e},

8) —NR^{d}R^{e},

9) —O(CR^{f}R^{g})ₙNR^{d}R^{e},

10) —C(O)R^{d},

11) —CO₂R^{d},

12) —CO₂(CR^{f}R^{g})ₙCONR^{d}R^{e},

13) —OC(O)R^{d},

14) —CN,

15) —C(O)NR^{d}R^{e},

16) —NR^{d}C(O)R^{e},

17) —OC(O)NR^{d}R^{e},

18) —NR^{d}C(O)OR^{e},

19) —NR^{d}C(O)NR^{d}R^{e},

20) —CR^{d}(N—OR^{e}),

21) —CF₃,

22) oxo

23) NR^{d}C(O)NR^{d}SO₂Rⁱ,

24) NR^{d}S(O)ₘR^{e},

25) —OS(O)₂OR^{d},

26) —OP(O)(OR^{d})₂; or

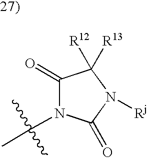

R^{y} is

1) a group selected from R^{x},

2) C1-10 alkyl,

3) C2-10 alkenyl,

4) C2-10 alkynyl,

5) aryl C1-10 alkyl,

6) heteroaryl C1-10 alkyl,

7) cycloalkyl,

8) heterocyclyl;

9) aryl; or

10) heteroaryl.

wherein alkyl, alkenyl, alkynyl, heteroaryl, and aryl are each optionally substituted with one to four substituents independently selected from R^{x};

Cy is

cycloalkyl, heterocyclyl, aryl, or heteroaryl;

m is an integer from 1 to 2;

n is an integer from 1 to 10;

X is

1) —C(O)OR^{d}

2) -5-tetrazolyl; or

3) —(CR^{f}R^{g})COOR^{d};

Y is

1) —S(O)₂—; or

2) —S(O)₂NR^{e}—

Z and A are independently selected from —C— and —C—C—;

B is selected from the group consisting of

1) a bond,

2) —C—,

3) —C—C—;

4) —C═C—,

5) a heteroatom selected from the group consisting of nitrogen, oxygen, and sulfur; and

6) —S(O)ₘ—.

These antagonists are useful in the treatment, prevention and suppression of diseases mediated by any VLA containing a β1 subunit. Such diseases include multiple sclerosis, asthma, allergic rhinitis, allergic conjunctivitis, inflammatory lung diseases, rheumatoid arthritis, septic arthritis, type I diabetes, organ transplantation, restenosis, autologous bone marrow transplantation, inflammatory sequelae of viral infections, myocarditis, inflammatory bowel disease including ulcerative colitis and Crohn's disease, certain types of toxic and immune-based nephritis, contact dermal hypersensitivity, psoriasis, tumor metastasis, atherosclerosis and fibrotic diseases. The antagonists may be particularly useful for cancer because of their anti-angiogenic properties.

### DETAILED DESCRIPTION OF THE INVENTION

The present compounds are biologically active small molecules and are generally composed of several domains: a) an acyl (including sulfonyl) moiety, b) a cyclic amino acid 1, and c) acid 2, and are named in a manner similar to that used to name oligopeptides.

Definitions:

“Antagonist” or “pan-β1 antagonist” are used interchangeably and includes any compound that inhibits (in any in vitro or in vivo system such as a cell, tissue, or tissue or cell culture) a “plurality” (defined below) of β1 subunit containing integrins from binding with an integrin ligand and/or receptor such as any receptor for the β1 subunit. For the purposes of the invention, a “pan-β1 antagonist” also refers to agents claimed herein which can inhibit or block integrin and/or integrin ligand-mediated binding or which can otherwise modulate integrin and/or integrin ligand function, e.g., by inhibiting or blocking integrin-ligand mediated integrin signal transduction. Such an antagonist of the integrin/integrin ligand interaction is an agent which has one or more of the following properties: (1) it coats, or binds to, a plurality of integrins (e.g, VLA-1 only or VLA-9 and VLA-1) on the surface of such integrin bearing or secreting cell with sufficient specificity to inhibit an integrin ligand/integrin interaction, e.g., the collagen/VLA-1 interaction; (2) it coats, or binds to, a plurality of integrins on the surface of an integrin-bearing or secreting cell with sufficient specificity to modify, and preferably to inhibit, transduction of an integrin-mediated signal e.g., collagen/VLA-1-mediated signaling; (3) it coats, or binds to, a plurality of integrin receptors, (e.g., collagen only or collagen and VCAM-1) in or on cells with sufficient specificity to inhibit the integrin/integrin ligand interaction; (4) it coats, or binds to, an integrin ligand (e.g., collagen) in or on cells with sufficient specificity to modify, and preferably to inhibit, transduction of integrin-mediated integrin signaling, e.g., collagen-mediated VLA-1 signaling.

In preferred embodiments the antagonist has one or both of properties 1 and 2. In other preferred embodiments the antagonist has one or both of properties 3 and 4. Moreover, more than one antagonist can be administered to a patient, e.g., an agent which binds to an integrin can be combined with an agent which binds to its ligand.

An antagonist of the invention has “biological activity” if it inhibits a “plurality” (defined below) of β1 subunit-containing integrins from binding with an integrin ligand and/or receptor such as any receptor for the β1 subunit, as determined by in vitro and in vivo tests known to workers having ordinary skill in the art.

“Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentynyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentenyl, 2-heptynyl and the like.

“Cycloalkyl” means mono- or bicyclic saturated carbocyclic rings, each of which having from 3 to 10 carbon atoms. The term also includes monocyclic rings fused to an aryl group in which the point of attachment is on the non-aromatic portion. Examples of cycloalkyl include cyclopropyl, cyclopentyl, cyclohexyl, cycloheptyl, tetrahydronaphthyl, decahydronaphthyl, indanyl, and the like.

“Aryl” means mono- or bicyclic aromatic rings containing only carbon atoms. The term also includes aryl group fused to a monocyclic cycloalkyl or monocyclic heterocyclyl group in which the point of attachment is on the aromatic portion. Examples of aryl include phenyl, naphthyl, indanyl, indenyl, tetrahydronaphthyl, 2,3 dihydrobenzofuranyl, benzopyranyl, 1,4-benzodioxanyl, and the like.

Heteroaryl” means a mono- or bicyclic aromatic ring containing at least one heteroatom selected from N, O and S, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, and the like.

“Heterocyclyl” means mono- or bicyclic saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “heterocyclyl” include pyrrolidinyl, piperidinyl, piperazinyl, imidazolidinyl, 2,3-dihydrofuro(2,3-b) pyridyl, benzoxazinyl, tetrahydrohydroquinolinyl, tetrahydroisoquinolinyl, dihydroindolyl, and the like. The term also includes partially unsaturated monocyclic rings that are not aromatic, such as 2- or 4 pyridones attached through the nitrogen or N-substituted-(1H,3H) pyrimidine-2,4-diones (N-substituted uracils).

“Halogen” includes fluorine, chlorine, bromine and iodine.

“Plurality” has a special meaning in the context of this disclosure since it is intended to mean: (I) a single β1 subunit-containing integrin provided that this integrin is not VLA-4; or (II) more than one β1 subunit-containing integrin such that an individual integrin may be VLA-4. Thus, the present methods utilize: (i) molecules capable of inhibiting any combination of two or more different beta1 containing integrins such as a method of antagonizing both VLA-4 (α4β1) and VLA-1 (α1β1) or VLA-2 (α2β1) and VLA-6 (α6β1) and so on; or (ii) molecules capable of inhibiting any single beta1 subunit-containing integrin provided that the single integrin is not VLA-4.

“Polymer” has its art recognized meaning as being a molecule constructed from many smaller structural units called “monomers”, bonded together (preferably covalently) in any pattern. The term includes linear molecules and branched molecules. The term also includes homopolymers where only one species of monomer is used to build the molecule, or copolymers where the molecule is composed of two different types of monomers and so on. Copolymers also include polymers where the distribution of monomers is random, alternating copolymers, block copolymers and graft copolymers.

Most preferably, the polymer is ‘biocompatible”. A “biocompatible” substance, as that term is used herein, is one that has no unacceptable toxic or injurious effects on biological function.

Antagonists of the invention are ‘small molecules’ which are organic molecules (i.e., having at least one alkyl or alkenyl carbon) although these molecules may, however, contain 1 or more peptide bonds, i.e., “peptdomimetics”. Nevertheless, peptides and proteins themselves are not included within the scope of the invention. A “small molecule”, as defined herein, has a molecular weight generally less than about 2000.

The term “effective amount” as used herein, means an amount of a compound of the present invention which inhibits a “plurality” (defined herein) of β1 subunit containing integrins from binding with an integrin ligand and/or receptor, as determined by in vitro and in vivo tests known to workers having ordinary skill in the art.

Optical Isomers—Diastereomers—Geometric Isomers—Tautomers

Compounds of Formula I contain one or more asymmetric centers and can thus occur as racemates and racemic mixtures, single enantiomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers. Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent. Alternatively, any enantiomer of a compound of the general Formula I may be obtained by stereospecific synthesis using optically pure starting materials or reagents of known configuration.

Other Modifications of the Antagonists

Other species are within the scope of the generic formulae I are described herein. For example, an exemplary series of pan beta 1 antagonists is found in Formula 4:

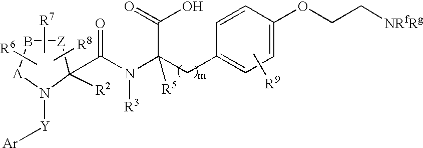

wherein wherein Ar is aryl, heteroaryl, aryl-substituted aryl, aryl substituted heteroaryl which are optionally substituted with one to four substituents independently selected from R^{x} and R⁹ is selected from H and R^{y} and all other constituents are as described herein.

Another embodiment of the invention are the hydantoin derivatives as set forth in Formula 5:

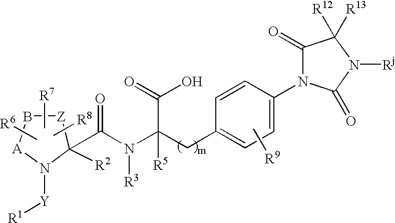

wherein R⁹ is selected from H and R^{y} and all other substituents are as described herein.

Other hydantoin derivatives are embodied by Formula 6:

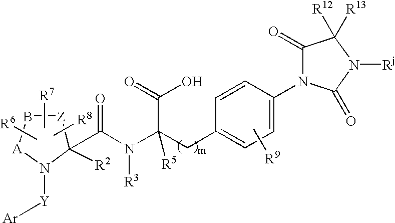

where Ar is aryl, heteroaryl, aryl-substituted aryl, aryl substituted heteroaryl which are optionally substituted with one to four substituents independently selected from R^{x}, R⁹ is selected from H and R^{y} and all other substituents are as described herein.

Specific structures intended to fall within the scope of the present invention are listed in Table 1 along with their mass as determined on either a Platform LCZ mass spectrometer (electrospray positive) or a VG Platform II mass spectrometer (electrospray positive or negative).

| | structure | mass spec |
|---|---|---|
| 1 | 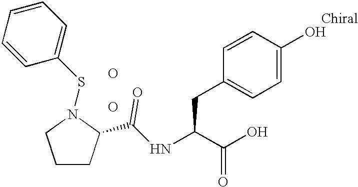 | 419.00 (M + 1) |
| 2 | 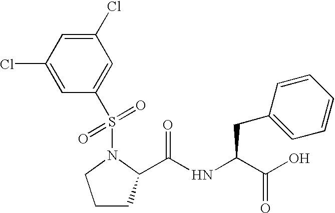 | 471 (M + !) |
| 3 | 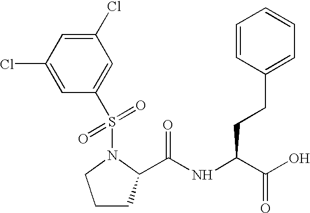 | 485 (M + 1) |
| 4 | 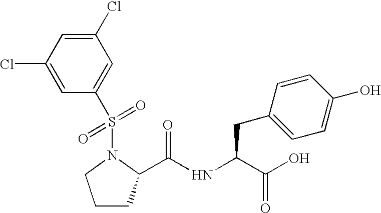 | 486.9 (M + 1) |
| 5 | 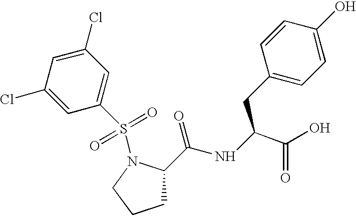 | 500.3 (M − 1) |
| 6 | 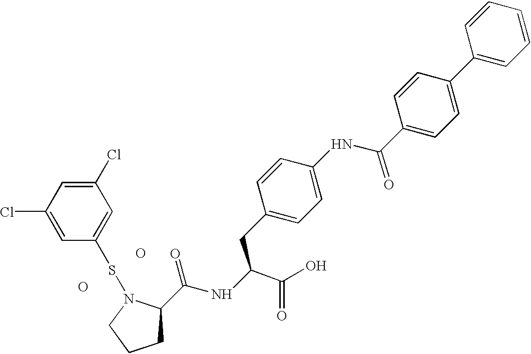 | 667.82 (M + 1) |
| 7 | 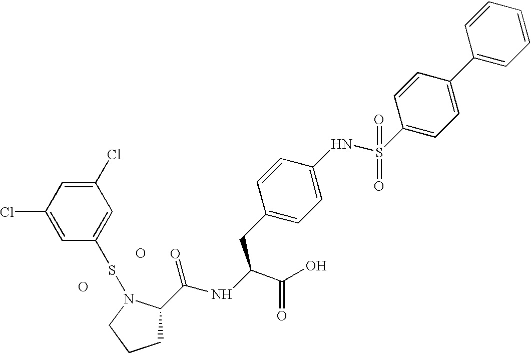 | 701.76 (M + 1) |
| 8 | 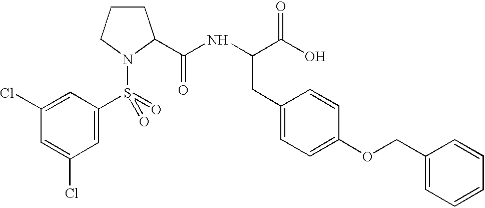 | 602.2 (M + 1) |
| 9 | 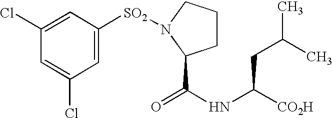 | 436.91 (M + 1) |
| 10 | 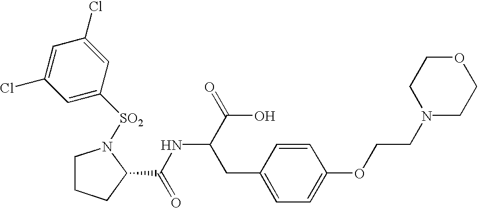 | 600.4 (M + 1) |
| 11 | 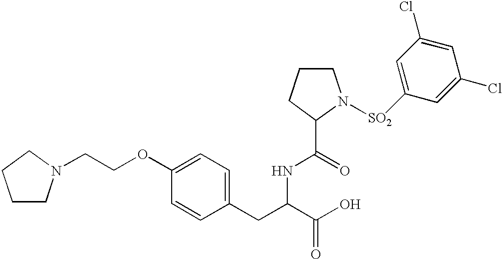 | 584.4 (M + 1) |
| 12 | 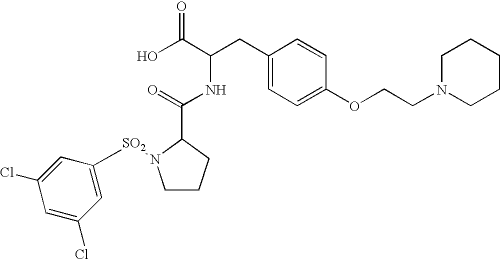 | 598.03 (M + 1) |
| 13 | 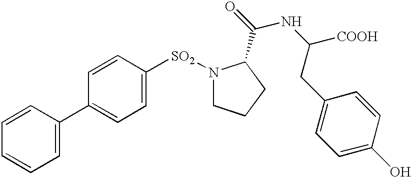 | 495.08 (M + 1) |
| 14 | 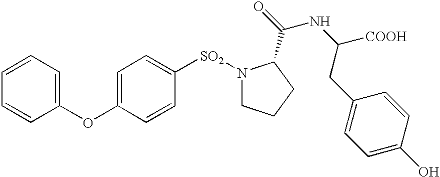 | 511.17 (M + 1) |
| 15 | 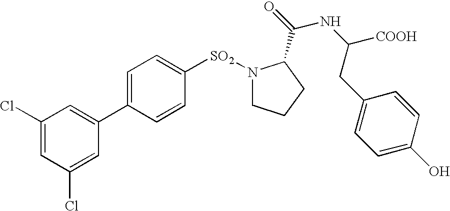 | 562.96 (M + 1) |
| 16 | 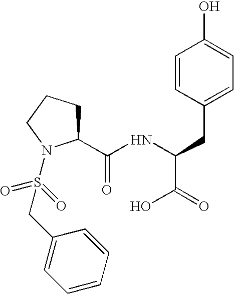 | 502.08 (M + 1) |
| 17 | 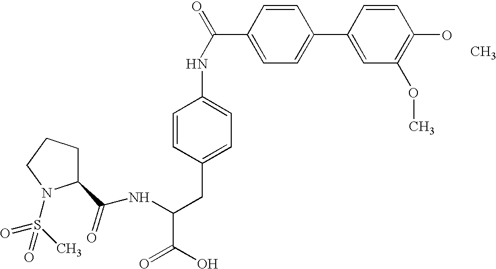 | 596.05 (M + 1) |
| 18 | 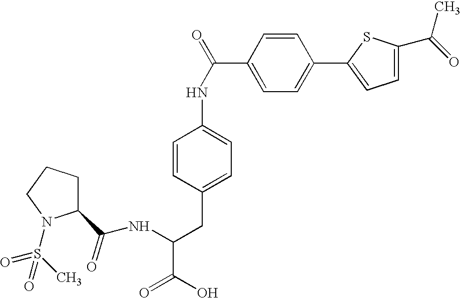 | 584.03 (M + 1) |
| 19 | 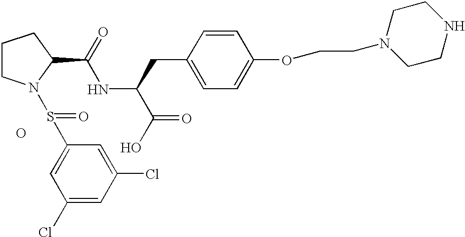 | 600.94 (M + 1) |
| 20 | 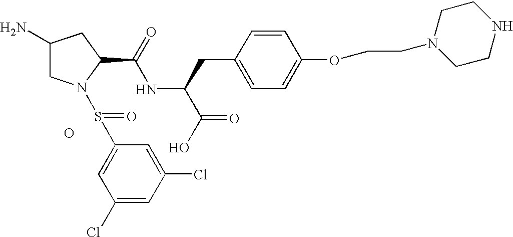 | 614.02 (M + 1) |
| 21 | 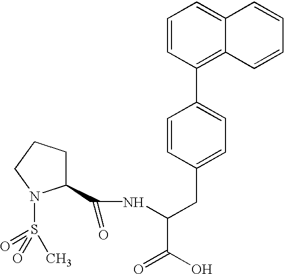 | 465.07 (M − 1) |
| 22 | 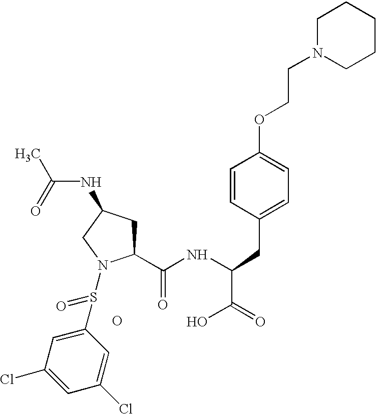 | 656.96 (M + 1) |
| 23 | 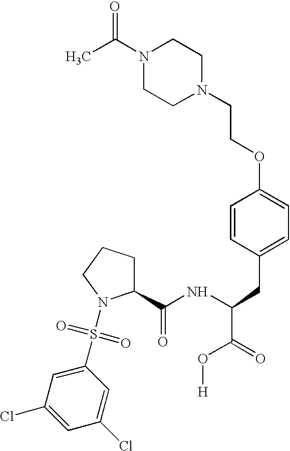 | 641.12 (M + 1) |
| 24 | 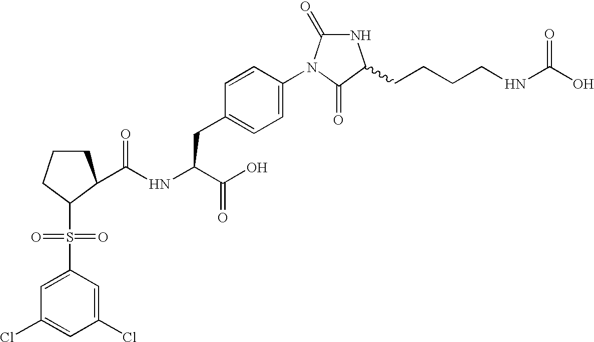 | 683.93 (M + 1) |
| 25 | 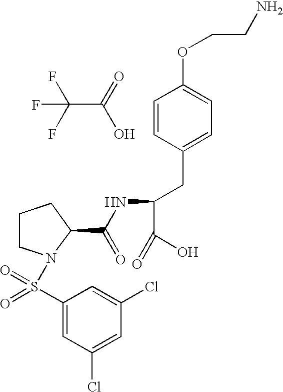 | 530.01 (M + 1) |
| 26 | 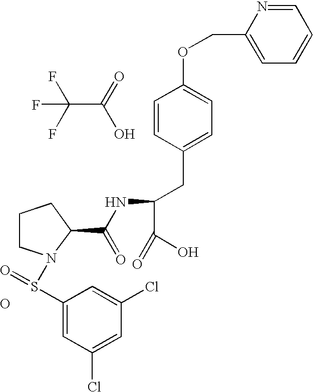 | 577.96 (M + 1) |
| 27 | 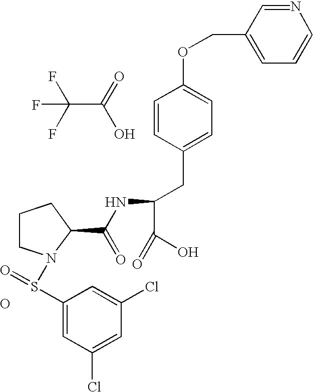 | 577.95 (M + 1) |
| 28 | 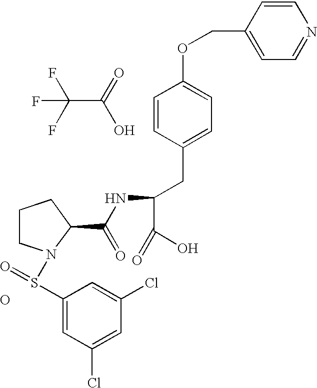 | 577.94 (M + 1) |
| 29 | 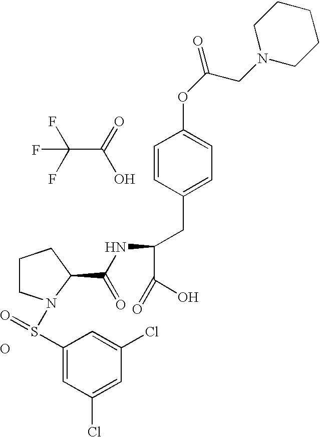 | 610.95 (M + 1) |
| 30 | 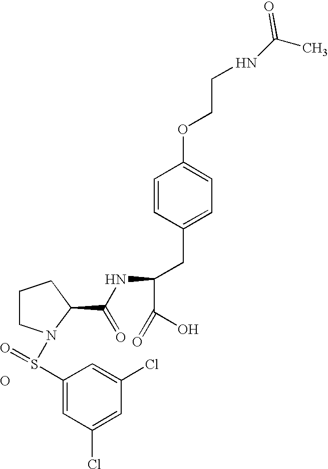 | 573.02 (M + 1) |
| 31 | 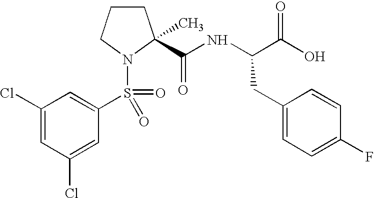 | 502.95 (M) |
| 32 | 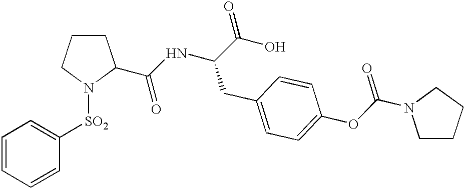 | 516.97 (M + 1) |
| 33 | 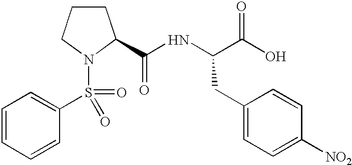 | 445.75 (M − 1) |
| 34 | 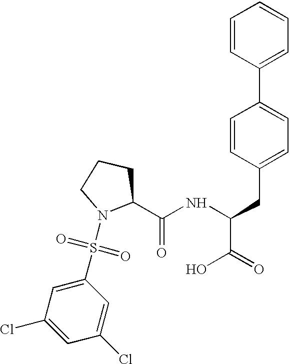 | 546.90 (M + 1) |
| 35 | 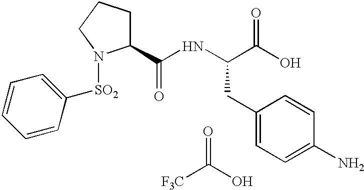 | 415.84 (M + 1) |
| 36 | 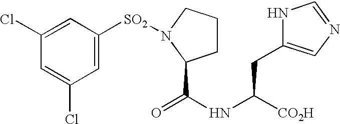 | 4650.93 (M + 1) |
| 37 | 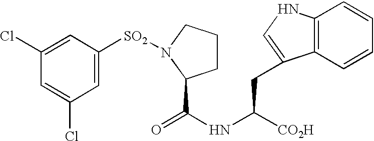 | 458.05 (M + 1) |
| 38 | 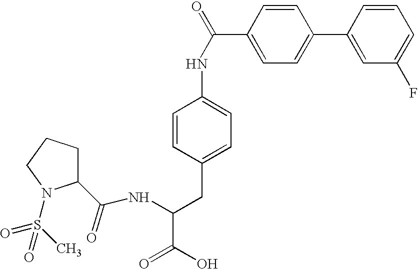 | 554.10 ((M + 1) |
| 39 | 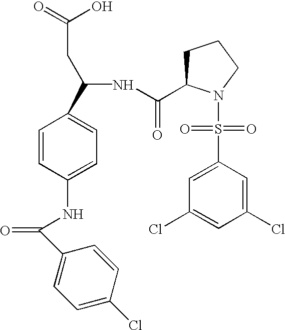 | 637.6 (M − 1) |
| 40 | 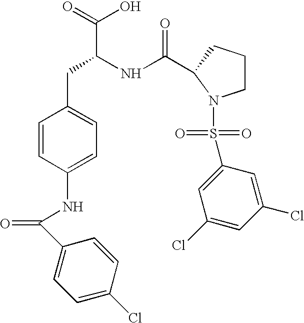 | 637.55 (M − 1) |
| 41 | 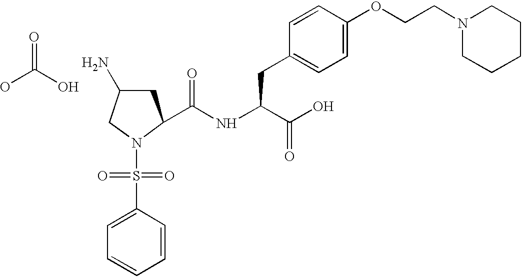 | 578.97 (M + 1) |
| 42 | 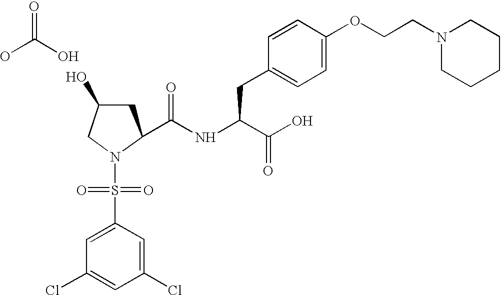 | 613.96 (M + 1) |
| 43 | 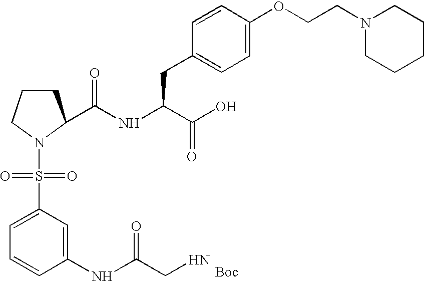 | 703.17 (M + 1) |
| 44 | 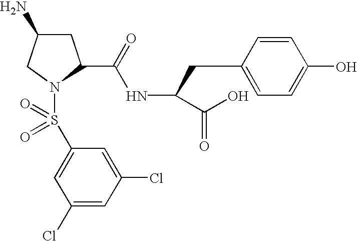 | 503.86 (M + 1) |

Polymer Conjugate Forms

Within the broad scope of the present invention, a single polymer molecule may be employed for conjugation with a pan beta1 integrin antagonist, although it is also contemplated that more than one polymer molecule can be attached as well. Conjugated pan beta1 integrin antagonist compositions of the invention may find utility in both in vivo as well as non-in vivo applications. Additionally, it will be recognized that the conjugating polymer may utilize any other groups, moieties, or other conjugated species, as appropriate to the end use application. By way of example, it may be useful in some applications to covalently bond to the polymer a functional moiety imparting UV-degradation resistance, or antioxidation, or other properties or characteristics to the polymer. As a further example, it may be advantageous in some applications to functionalize the polymer to render it reactive and enable it to cross-link to a drug molecule, to enhance various properties or characteristics of the overall conjugated material. Accordingly, the polymer may contain any functionality, repeating groups, linkages, or other constitutent structures which do not preclude the efficacy of the conjugated pan beta1 integrin antagonist composition for its intended purpose. Other objectives and advantages of the present invention will be more fully apparent from the ensuing disclosure and appended claims.

Illustrative polymers that may usefully be employed to achieve these desirable characteristics are described below in exemplary reaction schemes. In one embodiment of a covalently bonded antagonist/polymer conjugate, the polymer is coupled to the antagonist (preferably via a linker moiety) to form stable bonds that are not significantly cleavable by human enzymes. Generally, for a bond to be not ‘significantly’ cleavable requires that no more than about 20% of the bonds connecting the polymer and the antagonist(s) to which the polymer is linked, are cleaved within a 24 hour period, as measured by standard techniques in the art including, but not limited to, high pressure liquid chromatography (HPLC).

Pan beta1 integrin antagonists are conjugated most preferably via a terminal reactive group on the polymer although conjugations can also be branched from non-terminal reactive groups. The polymer with the reactive group(s) is designated herein as “activated polymer”. The reactive group selectively reacts with reactive groups on the antagonist molecule. The activated polymer(s) is reacted so that attachment may occur at any available pan beta1 integrin antagonist functional group. Amino, carbon, free carboxylic groups, suitably activated carbonyl groups, hydroxyl, guanidyl, oxidized carbohydrate moieties, amino, carbon and mercapto groups of the pan beta1 integrin antagonist (if available) can be used as attachment sites.

Generally from about 1.0 to about 10 moles of activated polymer per mole of antagonist, depending on antagonist concentration, is employed. The final amount is a balance between maximizing the extent of the reaction while minimizing non-specific modifications of the product and, at the same time, defining chemistries that will maintain optimum activity, while at the same time optimizing, if possible, the half-life of the antagonist. Preferably, at least about 50% of the biological activity of the antagonist is retained, and most preferably 100% is retained.

The reactions may take place by any suitable art-recognized method used for reacting biologically active materials with inert polymers. Generally the process involves preparing an activated polymer and thereafter reacting the antagonist with the activated polymer to produce the soluble compound suitable for formulation. The above modification reaction can be performed by several methods, which may involve one or more steps.

The polymeric substances included herein are preferably water-soluble at room temperature. A non-limiting list of such polymers includes polyalkylene oxide homopolymers such as polyethylene glycol (PEG) or polypropylene glycols, polyoxyethylenated polyols, copolymers thereof and block copolymers thereof, provided that the water solubility of the block copolymers is maintained.

In the preferred practice of the present invention, polyalkylene glycol residues of C1-C4 alkyl polyalkylene glycols, preferably polyethylene glycol (PEG), or poly(oxy)alkylene glycol residues of such glycols are advantageously incorporated in the polymer systems of interest. Thus, the polymer to which the pan beta1 antagonist is attached can be a homopolymer of polyethylene glycol (PEG) or is a polyoxyethylated polyol, provided in all cases that the polymer is soluble in water at room temperature. Non-limiting examples of such polymers include polyalkylene oxide homopolymers such as PEG or polypropylene glycols, polyoxyethylenated glycols, copolymers thereof and block copolymers thereof, provided that the water solubility of the block copolymer is maintained. Examples of polyoxyethylated polyols include, for example, polyoxyethylated glycerol, polyoxyethylated sorbitol, polyoxyethylated glucose, or the like. The glycerol backbone of polyoxyethylated glycerol is the same backbone occurring naturally in, for example, animals and humans in mono-, di-, and triglycerides. Therefore, this branching would not necessarily be seen as a foreign agent in the body.

A general formula for PEG and its derivatives is R″—(CH₂CH₂O)[x]—(CH₂)[y]—R′, where (x) represents the degree of polymerization or number of repeating units in the polymer chain and is dependent on the molecular weight of the polymer, (y) represents a positive integer, R′ is (CHR¹), where R¹is as defined in claim 1 and R″ is a capping group (including, without limitation, OH, C[1-4]alkyl moieties, or various biologically active and inactive moieties) or is R′. In particular, polyethylene glycols (PEG's), mono-activated, C[1-4]alkyl-terminated PAO's such as mono-methyl-terminated polyethylene glycols (mPEG's) are preferred when mono- substituted polymers are desired; bis-activated polyethylene oxides are preferred when disubstituted antagonists are desired.

As an alternative to polyalkylene oxides, dextran, polyvinyl pyrrolidones, polyacrylamides, polyvinyl alcohols, carbohydrate-based polymers and the like may be used.

Those of ordinary skill in the art will recognize that the foregoing list is merely illustrative and that all polymer materials having the qualities described herein are contemplated. The polymer need not have any particular molecular weight, but it is preferred that the molecular weight be between about 300 and 100,000, more preferably between 10,000 and 40,000. In particular, sizes of 20,000 or more are best at preventing loss of the product due to filtration in the kidneys.

Polyalkylene glycol derivatization has a number of advantageous properties in the formulation of polymer-pan beta1 integrin antagonist conjugates in the practice of the present invention, as associated with the following properties of polyalkylene glycol derivatives: improvement of aqueous solubility, while at the same time eliciting no antigenic or immunogenic response; high degrees of biocompatibility; absence of in vivo biodegradation of the polyalkylene glycol derivatives; and ease of excretion by living organisms.

Polyethylene glycol (PEG) and related polyalkylene oxides (PAO's) are known in the art as being useful adjuncts for the preparation of drugs. See for example, PCT WO 93/24476. PEG has also been conjugated to proteins, peptides and enzymes to increase aqueous solubility and circulating life in vivo as well as reduce antigenicity. See, for example, U.S. Pat. Nos. 5,298,643 and 5,321,095, both to Greenwald, et al. PCT WO 93/24476 discloses using an ester linkage to covalently bind an organic molecule to water-soluble polyethylene glycols.

In one aspect of the invention, one can utilize a pan beta1 integrin antagonist covalently bonded to the polymer component in which the nature of the conjugation involves one or more noncleavable covalent chemical bonds which, preferably, are resistant to degradation by human enzymes. For instance, Greenwald et al., supra, disclose biologically-active conjugates having substantially hydrolysis-resistant bonds (linkages) between a polyalkylene oxide and the target moiety. One example of a noncleavable linker suitable for the pan beta 1 antagonists of the present invention is:

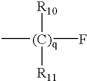

wherein R₁₀ and R₁₁ are independently selected from the group consisting of H, C₁₋₆ alkyls, aryls, substituted aryls, aralkyls, heteroalkyls, substituted heteroalkyls and substituted C₁₋₆ alkyls, q is a positive integer and F is selected from O, NR¹, S, SO, SO₂.

In another embodiment, the linkages between a polymer and the pan beta 1 antagonist of the invention is cleavable, allowing for control in terms of the time course over which the polymer may be cleaved from the pan beta1 integrin antagonist. This covalent bond between the pan beta1 integrin antagonist and the polymer may be cleaved by chemical or enzymatic reaction. In order to provide a hydrolyzable linkage, mono- or di-acid activated polymers such as PEG acids or PEG diacids are used. Suitable PAO acids can be synthesized by converting mPEG-OH to an ethyl ester. See also Gehrhardt, H., et al. Polymer Bulletin 18: 487 (1987) and Veronese, F. M., et al., J. Controlled Release 10; 145 (1989). Alternatively, the PAO-acid can be synthesized by converting mPEG-OH into a t-butyl ester. Ohya, et al., J. Bioactive and Compatible Polymers Vol. 10 January, 1995, 51-66, disclose doxorubicin-PEG conjugates which are prepared by linking the two substituents via various linkages including esters. It will be clear from the foregoing that other polyalkylene oxide derivatives of the foregoing, such as the polypropylene glycol acids, POG acids, etc., as well as other bifunctional linking groups are also contemplated. The polymer-pan beta1 integrin antagonist product retains an acceptable amount of activity. Concurrently, portions of polyethylene glycol are present in the conjugating polymer to endow the polymer-pan beta1 integrin antagonist conjugate with high aqueous solubility and prolonged blood circulation capability.

It is to be understood that the reaction schemes described herein are provided for the purposes of illustration only and are not to be limiting with respect to the reactions and structures which may be utilized in the modification of the pan beta1 integrin antagonist, e.g., to achieve solubility, stabilization, and cell membrane affinity for parenteral and oral administration. The activity and stability of the pan beta1 integrin antagonist conjugates can be varied in several ways by using a polymer of different molecular size. Solubilities of the conjugates can be varied by changing the proportion and size of the polyethylene glycol fragment incorporated in the polymer composition.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N, N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

Utilities

The ability of the compounds of Formula I to antagonize the actions of any VLA integrin containing a β1 subunit makes them useful for preventing or reversing the symptoms, disorders or diseases induced by the binding of VLA to its various ligands. Thus, these antagonists will inhibit cell adhesion processes including cell activation, migration, proliferation and differentiation and be useful in conditions such as acute or chronic renal failure or acute brain injury. Accordingly, another aspect of the present invention provides a method for the treatment (including prevention, alleviation, amelioration or suppression) of diseases or disorders or symptoms, including fibrotic conditions and an inflammatory disorder mediated by β1 integrin binding and cell adhesion activation, which comprises administering to a mammal an effective amount of a compound of Formula I.

As used herein, “an inflammatory disorder”, includes, but is not limited to, skin related conditions such as psoriasis, eczema, burns and dermatitis. Other inflammatory disorders contemplated for treatment by the methods of the present invention include, but are not limited to the treatment of asthma, bronchitis, menstrual cramps, tendinitis, bursitis, and the treatment of pain and headaches, or as an antipyretic for the treatment of fever. The methods of the invention also would be useful to treat gastrointestinal conditions such as inflammatory bowel disease, Crohn's disease, gastritis, irritable bowel syndrome and ulcerative colitis and for the prevention of colorectal cancer. The methods of the invention would be useful in treating inflammation in such diseases as vascular diseases, migraine headaches, periarteritis nodosa, thyroiditis, aplastic anemia, Hodgkin's disease, rheumatic fever, type I diabetes, myasthenia gravis, multiple sclerosis, sarcoidosis, nephrotic syndrome, Behcet's syndrome, polymyositis, gingivitis, hypersensitivity, conjunctivitis, swelling occurring after injury, myocardial ischemia, and the like. The methods of the invention would also be useful in the treatment of allergic rhinitis, respiratory distress syndrome, endotoxin shock syndrome, and atherosclerosis as well as asthma, allergic rhinitis, allergic conjunctivitis, inflammatory lung diseases, rheumatoid arthritis, septic arthritis, organ transplantation rejection, restenosis, autologous bone marrow transplantation, inflammatory sequelae of viral infections, myocarditis, tumor metastasis, and atherosclerosis.

Pan-β1 antagonists of the present invention may also be useful in treating a subject with a fibrotic condition. The term “fibrotic condition” refers to, but is not limited to, subjects afflicted with fibrosis of an internal organ, subjects afflicted with a dermal fibrosing disorder, and subjects afflicted with fibrotic conditions of the eye.

Fibrosis of internal organs (e.g., liver, lung, kidney, heart blood vessels, gastrointestinal tract) occurs in disorders such as pulmonary fibrosis, myelofibrosis, liver cirrhosis, mesangial proliferative glomerulonephritis, crescentic glomerulonephritis, diabetic nephropathy, renal interstitial fibrosis, renal fibrosis in patients receiving cyclosporin, and HIV associated nephropathy. Dermal fibrosing disorders include, but are not limited to, scleroderma, morphea, keloids, hypertrophic scars, familial cutaneous collagenoma, and connective tissue nevi of the collagen type. Fibrotic conditions of the eye include conditions such as diabetic retinopathy, postsurgical scarring (for example, after glaucoma filtering surgery and after cross-eye surgery), and proliferative vitreoretinopathy. Additional fibrotic conditions which may be treated by the methods of the present invention include: rheumatoid arthritis, diseases associated with prolonged joint pain and deteriorated joints; progressive systemic sclerosis, polymyositis, dermatomyositis, eosinophilic fascitis, morphea, Raynaud's syndrome, and nasal polyposis.

In addition, fibrotic conditions which may be treated the methods of present invention also include inhibiting overproduction of scarring in patients who are known to form keloids or hypertrophic scars, inhibiting or preventing scarring or overproduction of scarring during healing of various types of wounds including surgical incisions, surgical abdominal wounds and traumatic lacerations, preventing or inhibiting scarring and reclosing of arteries following coronary angioplasty, preventing or inhibiting excess scar or fibrous tissue formation associated with cardiac fibrosis after infarction and in hypersensitive vasculopathy.

Testing Antagonists of the Invention for Function

In vitro Testing

The cell adhesion inhibitory activity of these compounds may be measured by determining the concentration of inhibitor required to block the binding of cells expressing beta 1 subunit containing integrins to extracellular matrix components such as collagen or fibronectin coated plates. In this assay microtiter wells are coated with, for example, collagen. Once the wells are coated, varying concentrations of the test compound are then added together with appropriately labeled, integrin-expressing cells. Alternatively, the test compound may be added first and allowed to incubate with the coated wells prior to the addition of the cells. The cells are allowed to incubate in the wells for at least 30 minutes. Following incubation, the wells are emptied and washed. Inhibition of binding is measured by quantitating the fluorescence or radioactivity bound to the plate for each of the various concentrations of test compound, as well as for controls containing no test compound.

Beta 1 subunit expressing cells that may be utilized in this assay include Ramos cells, Jurkat cells, A375 melanoma cells, as well as human peripheral blood lymphocytes (PBLs). These cells are commercially available and may be fluorescently or radioactively labeled if desired. A direct binding assay (“DBA”). may also be employed to quantitate the inhibitory activity of the compounds of this invention.

Generally, in vitro assays such as the adhesion inhibition and direct binding assays described above, substitute the appropriate integrin-expressing cell and corresponding ligand. For example, polymorphonuclear cells (PMNs) express integrins on their surface and bind to ICAM. Integrins are involved in platelet aggregation and inhibition may be measured in a standard platelet aggregation assay. VLA-5 binds specifically to Arg-Gly-Asp sequences, while VLA-6 binds to laminin.

In vivo Testing

Once pan beta1 antagonists are identified, they may be further characterized in in vivo assays, non-limiting examples of which are described below:

A. Contact Hypersensitivity

An exemplary animal model is described by P. L. Chisholm et al., “Monoclonal Antibodies to the Integrin α-4 Subunit Inhibit the Murine Contact Hypersensitivity Response”, Eur. J. Immunol., 23, pp. 682-688 (1993) and in “Current Protocols in Immunology”, J. E. Coligan, et al., Eds., John Wiley & Sons, New York, 1, pp. 4.2.1-4.2.5 (1991), the disclosures of which are herein incorporated by reference. In these assays, the skin of the animal is sensitized by exposure to an irritant, such as dinitrofluorobenzene, followed by light physical irritation, such as scratching the skin lightly with a sharp edge. Following a recovery period, the animals are re-sensitized following the same procedure. Several days after sensitization, one ear of the animal is exposed to the chemical irritant, while the other ear is treated with a non-irritant control solution. Shortly after treating the ears, the animals are given various doses of the pan beta 1 antagonists by subcutaneous injection. In vivo inhibition of cell adhesion-associated inflammation is assessed by measuring the ear swelling response of the animal in the treated versus untreated ear. Swelling is measured using calipers or other suitable instrument to measure ear thickness.

B. Delayed Hypersensitivity

SRBC-induced delayed type hypersensitivity (DTH) responses are adapted from the protocol of Hurtrel et al. 1992 Cell. Immunol. 142:252-263. Briefly, mice are immunized s.c. in the back with 2×10⁷ SRBC in 100 ul PBS on d 0. The mice are challenged on d 5 by injecting 1×10⁸ SRBC in 25 ul PBS s.c into the right hind footpad. Footpad thickness is measured with an engineer's caliper (Mitutoyo/MTI, Paramus, N.J.) 20 h after antigen challenge, and the degree of footpad swelling calculated. Results are reported as the mean percent increase footpad thickness±SEM and calculated as % increase=[1−(Right footpad thickness 20 h after antigen challenge/Uninjected left footpad thickness 20 h after antigen challenge)]×100. To block the effector phase of the SRBC-induced DTH response, antagonists of the invention which are prepared according to the methods described in the Examples are given prior to antigen challenge on d 5. SRBC-induced DTH is a well characterized in vivo model of inflammation, and in particular psoriasis, that has been used to demonstrate the importance of a variety of cytokines and adhesion molecules in inflammation (Tedder et al. 1995 J. Exp. Med. 181:2259-2264, Terashita et al. 1996 J. Immunol. 156:4638-4643).

In this manner, one may identify those inhibitors of this invention which are best suited for inhibiting inflammation.

C. Asthma

Another in vivo assay that may be employed to test the antagonists of this invention is the sheep asthma assay. This assay is performed essentially as described in W. M. Abraham et al., “β-Integrins Mediate Antigen-induced Late Bronchial Responses and Prolonged Airway Hyperresponsiveness in Sheep”, J. Clin. Invest., 93, pp. 776-87 (1994), the disclosure of which is herein incorporated by reference. This assay measures inhibition of Ascaris antigen-induced late phase airway responses and airway hyperresponsiveness in allergic sheep

D. Renal Failure

The agents of the present invention also may be tested in animal models of chronic renal failure. Mammalian models of chronic renal failure in, for example, mice, rats, guinea pigs, cats, dogs, sheep, goats, pigs, cows, horses, and non-human primates, may be created by causing an appropriate direct or indirect injury or insult to the renal tissues of the animal. Animal models of chronic renal failure may, for example, be created by performing a partial (e.g., 5/6) nephrectomy which reduces the number of functioning nephron units to a level which initiates compensatory renal hypertrophy, further nephron loss, and the progressive decline in renal function which characterizes chronic renal failure. The agents of the present invention may be evaluated for their therapeutic efficacy in causing a clinically significant improvement in a standard marker of renal function when administered to a mammalian subject (e.g., a human patient) in, or at risk of, chronic renal failure. Such markers of renal function are well known in the medical literature and include, without being limited to, rates of increase in BUN levels, rates of increase in serum creatinine, static measurements of BUN, static measurements of serum creatinine, glomerular filtration rates (GFR), ratios of BUN/creatinine, serum concentrations of sodium (Na+), urine/plasma ratios for creatinine, urine/plasma ratios for urea, urine osmolality, daily urine output, and the like (see, for example, Brenner and Lazarus (1994), in Harrison's Principles of Internal Medicine, 13th edition, Isselbacher et al., eds., McGraw Hill Text, New York; Luke and Strom (1994), in Internal Medicine, 4th Edition, J. H. Stein, ed., Mosby-Year Book, Inc. St. Louis.).

E. Acute Brain Injury

Male Sprague Dawley (SD) or spontaneously hypertensive rats (SHRS) are anesthetized using isoflurane and the right middle cerebral artery (MCAO) occluded by insertion of a 4-0 nylon monofilament up the internal carotid artery to the origin of the middle cerebral artery (MCA) (Zea Longa et al, 1989 Stroke 20:84). After 1 h the filament is retracted, the ischemic territory reperfused and the animal allowed to recover. After 24 h the rats are sacrificed, at which time brains were removed and analyzed histologically to quantify infarct volume.

Groups of animals are treated with either vehicle (PBS) or antagonist of the invention by continuous subcutaneous infusion via osmotic mini-pumps. Primed mini osmotic pumps (Alza Corp.,) are implanted into the subcutaneous space at the scruff of the neck immediately prior to induction of cerebral ischemia. The pumps are loaded to release antagonist.

F. Fibrosis

For vessel injury leading to fibrosis, male Sprague-Dawley rats weighing 400 g and about 3-4 months of age (Bantin & Kingman, Edwards, Wash.) are used. The left common carotid artery is denuded with a 2 F balloon catheter by introducing the catheter through the external carotid artery. The distal left common carotid and external carotid arteries are exposed through a midline wound in the neck. The catheter is passed three times with the balloon distended sufficiently with saline to generate slight resistance; this method produces distension of the carotid itself. the external carotid is ligated ater removal of the catherter and the wound closed. Experimental treatments include a series of injections of antagonist given every other day (post-operation). After 14 days post balloon catheter denudation, all rats are anesthetized and the carotid arteries fixed by perfusion at 120 mm Hg pressure with 1% paraformaldehyde and 1.25% glutaraldehyde in 0.1M phosphate buffer, pH 7.4 via a large cannula placed retrograde in the abdominal aorta. Ten minutes before fixation, these animals are given an intravenous injection of Evans blue (0.3 ml in 5% saline solution). After 5 minutes of perfusion, the entire left and right common carotid arteries are retrieved, including the aortic arch. The vessels are further fixed by immersion in the same fixative as was used for perfusion. Arterial segments are assayed for the presence or absence of endothelium by obtaining three segments from the denuded, blue-stained left carotid artery and emdedding them in paraffin for cross sectioning using a “Micron” microtome. For measuring intimal areas, photomicrographs are obtained from 3-4 sections from each animal. The photomicrographs are digitized and anlaysed with image analysis software (NIH Image 1.55 for MacIntosh). Intimal areas are measured by determining the area between liumen and internal elastic lamina. Medial areas are determined by measuring the area between internal and external elastic lamina. Intimal/medial area ratios are calculated from the measurements.

For testing the effect of the present antagonists on lung fibrosis, chronic respiratory disease-free Golden Syrain hamsters weighing 120-130 g are purchased from Charles River (Boston, Mass.) and housed in plastic cages in groups of 4 in facilities approved by the American Association for Accreditation of Laboratory Animal Care. The animals are allowed to acclimate for one week to laboratory conditions prior to starting the experiments. They have access to Rodent Laboratory Chow 5001 (Purina Mills, Inc., St. Louis, Mo.) and water ad libitum and housed in a room which gets the filtered air and has a 12 hr/12 hr light/dark cycle. Bleomycin sulfate is dissolved in pyrogen free sterile isotonic saline just before intratraceheal (IT) instillation. Under pentobarbital anesthesia (25-35 mg/kg ip) hamsters in appropriate groups receive either bleomycin (5.5 units/kg/4 ml) or an equivalent volume (4 ml/kg) of pyrogen free isotonic saline through transoral route. The antagonists of the invention are administered by intraperitoneal (IP) or intratrachial route at a therapeutic dose to hamsters in appropriate groups twice a week for 21-28 days post installation. Thereafter, the animals in each group are killed by an overdose of sodium pentobarbital (100-125 mg/kg ip) and their lungs processed for biochemical and histopathological studies.

G. Glomerulonephritis Model

Experimental glomerulonephritis is induced in rats with a single injection of anti-glomerular basement membrane nephrotixin serum (NTS), derived in rabbits. The experimental lesion is acute mesangial proliferative glomerulonephritis and is characterized by expansion of the mesangial matrix and hypercellularity. Of particular intererst, the nephritis reproducibly progresses through glomerular and tubulointerstitial scarring, to end stage renal disease.

First, glomerulonephritis is induced in rats by an intravenous injection of NTS serum. Next, for six days, two groups of rats are treated with daily intravenous injections of saline (the negative control group) or antagonists of the invention. On the tenth day, the animals are sacrificed and slides are made of the kidneys, which are stained with periodic acid-Schiff solution to emphasize the pathological changes. The extent of glomerular injury can be quantitated by performing glomerular cell counts from 30 randomly selected glomeruli from normal animals and nephritic animals in each group. Another measure of the effect of antagonists of the invention on the disease process is to quantitate the amount of extracellular matrix accumulation in the glomeruli. The degree of glomerular matrix expansion is determined as the percentage of each glomerulus occupied by the mesangial matrix according to the method of Raij et al. (1984) Kidney Int. 26: 137-43.

H. Arthritis Model

Arthritis is induced in pathogen-free female LEW rats (Harlan Sprague Dawley, Indianapolis, Ind.) weighing about 100 grams. Each receives a dose of cell wall fragments from Group A streptococci (SCW) (30 mu g rhamnose/gm bodyweight), injected intraperitoneally (ip) according to the technique described in Brandes et al. (1991) J. Clin. Invest. 87:1108. SCW-injected and control LEW rats are given an intraarticular (IA) injection in one of the hind ankles of antagonists of the invention, carrier only, or a control.

Joints are clinically monitored by determining the amount of joint erythema, swelling and distortion on a scale of 0 (normal) to 4 (severe inflammation). Radiographs are taken and are evaluated for soft tissue swelling, joint space narrowing, bone erosions and deformity. Tissue specimens are obtained and prepared for histopathologic analysis as described in Brandes et al., ibid. Total RNA is isolated from excised synovial tissues according to the method of Allen et al. (1990) J. Exp. Med. 171:231. Other models are available. See Terato et al. 1992 J. Immunol. 148:2103-2108; Terato et al. 1995 Autoimmunity. 22:137-147. Briefly, arthrogen-CIA Antibody kits are purchased from Stratagene (La Jolla, Calif.) and arthritis induced using the Terato et al. protocol. Briefly, arthritis is induced through i.p. injection of a cocktail of 4 anti-collagen type II mAbs (1 mg each) on d 0, followed by i.p. injection of 50 ug LPS on d 3. Over the course of the next 3-4 d, the mice develop swollen wrists, ankles and digits. Therapeutic or control antagonist is administered i.p. 4 h prior to injection of the anti-collagen mAbs on d 0, and again 4 h prior to LPS administration on d 3, and then continuing every 3^{rd} day for the length of the experiment. Beginning on d 3, mice are evaluated for the development of arthritis. Severity of arthritis in each limb is scored using a four point system. 0=normal; 1=mild redness, slight swelling of ankle or wrist; 2=moderate swelling of ankle or wrist; 3=severe swelling including some digits, ankle, and foot; 4=maximally inflamed.

Dose Ranges

The magnitude of prophylactic or therapeutic dose of a compound of Formula I will, of course, vary with the nature of the severity of the condition to be treated and with the particular compound of Formula I and its route of administration. It will also vary according to the age, weight and response of the individual patient. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

For use where a composition for intravenous administration is employed, a suitable dosage range is from about 0.001 mg to about 25 mg (preferably from 0.01 mg to about 1 mg) of a compound of Formula I per kg of body weight per day and for cytoprotective use from about 0.1 mg to about 100 mg (preferably from about 1 mg to about 100 mg and more preferably from about 1 mg to about 10 mg) of a compound of Formula I per kg of body weight per day. In the case where an oral composition is employed, a suitable dosage range is, e.g. from about 0.01 mg to about 100 mg of a compound of Formula I per kg of body weight per day, preferably from about 0.1 mg to about 10 mg per kg and for cytoprotective use from 0.1 mg to about 100 mg (preferably from about 1 mg to about 100 mg and more preferably from about 10 mg to about 100 mg) of a compound of Formula I per kg of body weight per day.

Pharmaceutical Compositions

Another aspect of the present invention provides pharmaceutical compositions which comprises a compound of Formula I and a pharmaceutically acceptable carrier.

The term “composition”, as in pharmaceutical composition, is intended to encompass a product comprising the active ingredients, and the inert ingredients (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredients, and pharmaceutically acceptable excipients.

Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well known in the art of pharmacy. For administration by inhalation, the compounds of the present invention are conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, ointments, lotions, dusting powders, and the like. In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent, the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Desirably, each tablet contains from about 1 mg to about 500 mg of the active ingredient and each cachet or capsule contains from about 1 to about 500 mg of the active ingredient.

The following are examples of representative pharmaceutical dosage forms for the compounds of Formula 1:

| Injectable Suspension (i.m.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

Water for injection to a total volume of 1 mL

| Tablet | mg/tablet |
|---|---|
| Compound of Formula 1 | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

| Aerosol | Per canister | |
|---|---|---|
| Compound of Formula I | 24 | mg |
| Lecithin, NF Liquid Concentrate | 1.2 | mg |
| Trichlorofluoromethane, NF | 4.025 | g |
| Dichlorodifluoromethane, NF | 12.15 | g |

Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) VLA-4 antagonists such as those described in U.S. Pat. No. 5,510,332, W097/03094, W097/02289P, W096t4O781P, W096/22966, W096/20216, W096101644, W096/06108, W095/15973 and W096131206; (b) steroids such as declomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratacline, cetirizine, fexofenadine, descarboethoxyloratadine, and the like; (e) non-steroidal anti-asthmatics such as P2-agonists (terbutaline, metaproterenol).

### EXAMPLES

The following examples serve to provide further appreciation of the invention but are not meant in any way to restrict the effective scope of the invention. The numbers shown in bold in the Examples correspond to the compounds shown in the schematic diagrams set forth in the accompanying Table 1.

In general, an amide bond is formed between a proline derivative and a H-Tyr(Bu)OR. A sulfonyl amide is formed, followed by deprotection of the phenol, alkylation of the phenol and then hydrolysis of the ester moiety. In some cases the phenol is not alkylated. In other cases, the phenol is alkylated with 2-chloro-bromoethane. The bromide can then be displaced with the appropriate amine. The carbamate of the phenol may also be formed using the appropriate aminocarbonyl chloride.

In another scheme, an amide bond is formed between a proline derivative and a nitrophenylalanine derivative on resin. A sulfonyl amide is formed, followed by reduction of the nitro group and acylation of the resulting amine with iodobenzoic acid. Suzuki coupling of a boronic ester with the aryl iodide is followed by cleavage from the resin. Alternatively, the iodophenylalanine derivative is attached to resin and acylated with proline. A sulfonyl amide is formed, Suzuki coupling of a boronic ester with the aryl iodide and cleavage from the resin completes the sequence. The nitro phenylalanine derivative may be attached to the resin, the nitro group reduced and acylated with bromoacetic acid. The bromine can then be displaced with a secondary amine. An amide bond can be formed with a proline derivative, the proline nitrogen sulfonated and the compound cleaved from the resin. Alternatively, an amide bond with proline and sulfonation occurs before reduction of the nitro group. Acylation of the aniline with a protected aminoacid, deprotection of the aminoacid, formation of the carbamate with nitrophenyl chloroformate and cyclization with base forms the hydantion which can be cleaved from the resin. Alternatively, the aniline may be acylated or sulfonated prior to cleavage from the resin. In another example, a protected aminoacid is attached to the resin, the amine deprotected, sulfonated and the compound cleaved from the resin.

An appropriate hydroxyproline can be bis-sulfonated and the sulfonic ester displaced with sodium azide. Following hydrolysis of the ester moiety, the carboxylic acid is coupled to an appropriate phenylalanine derivative. The azide is reduced to the amine, which may or may not be acylated, and the ester moiety hydrolyzed. Alternatively, the ester of the bis-sulfonated hydroxyproline derivative may be hydrolyzed and coupled with an appropriate phenylalanine derivative. Protecting groups are then removed.

All final products are purified via reverse phase HPLC using water/acetonitrile gradients on a C18 column. Mass specral data is determined on either a Platform LCZ mass spectrometer (electrospray positive) or a VG Platform II mass spectrometer (electrospray positive or negative).

### Example 1

Cell Adhesion Assay Protocol

This illustrates the protocol for determining utility of the antagonists listed in Table 1. More particularly, the protocol determines the ability of such organic compounds to inhibit and prevent collagen-based cell adhesion.

Protocol:

1. Coat a 96-well plate with Collagen IV (0.5 ug/ml), Collagen I (5 ug/ml), BSA-CS1 (1 ug/ml), or Laminin (20 ug/ml) for adhesion assays, α1β1, α2β1, α4β1, or α6β1, respectively, in NaBicarb. pH 9.2 at 4° C. overnight.

2. Wash the plate twice with 1×PBS, 100 ul/well.

3. Block the plate with 1% heat-treated BSA in PBS, 100 ul/well for 1+ hr.

4. Wash the plate twice with assay buffer (TBS complete+1 mM MnCl₂), 100 ul/well.

5. Add compound (2×desired conc.) and cells (4×10⁶ cells/ml, labeled with BCECF.AM [2′,7′-bis(2-carboxyethyl)-5-(-6)-carboxyfluorescein, acetoxymethyl ester] at 37° C. for 15 min.) each at 25 ul/well.

6. Incubate the plate at room temperature for 30 minutes.

7. Before washing the plate, read the total cells input in a fluorescent plate reader.

8. Wash the plate three times with assay buffer, 100 ul/well.

9. Read the remaining bound cells in the fluorescent plate reader.

Results:

Of the 44 compounds tested, 35 (about 75%) inhibited all four integrins tested (VLA-1, VLA-2, VLA-4 and VLA-6) with IC50 values (the concentration that gives 50% reduction in binding as measured in the above assay) ranging from 4 nanomolar to 70 micromolar. The remaining 9 compounds inhibited less than all four integrins in various combinations.

### Example 2

General Procedure for Synthesis of Compounds 1, 4, 13, 14, 15, 16

Step 1

Wang resin loaded with Fmoc-Tyr(But)-OH (0.22 mmol) was treated with 25% piperidine in DMF (2×10 min) and then washed with DMF (3×), iPrOH (3×) and CH₂Cl₂ (5×). The resin was then agitated with proline (2.2 mmol), diisopropyl carbodiimide (1.3 mmol) and HOBT (1.3 mmol) in NMP overnight. The resin was then filtered and washed with NMP (3×), iPrOH (3×) and CH₂Cl₂ (5×).

Step 2

The resin (0.22 mmol) was agitated with the appropriate sulfonyl chloride (2.2 mmol) in pyridine overnight, filtered and then washed with NMP (3×), iPrOH (3×) and CH₂Cl₂ (5×).

Step 3

The product was cleaved from the resin with 95:5 trifluoroacetic acid/water (30 min), filtered, concentrated in vacuo and purified via reverse phase HPLC (acetonitrile/water).

### Example 3

General Procedure for Synthesis of Compounds 17, 18, 33, 35, 38

Step 1

Wang resin loaded with N-Fmoc-p-nitrophenylalanine (0.83 mmol) was treated with 25% piperidine in DMF (2×10 min) and then washed with DMF (3×), iPrOH (3×) and CH₂Cl₂ (5×). The resin was then agitated with proline (5 mmol), diisopropyl carbodiimide (5 mmol) and HOBT (5 mmol) in NMP overnight. The resin was then filtered and washed with NMP (3×), iPrOH (3×) and CH₂Cl₂ (5×).

Step 2

The resin (0.83 mmol) was agitated with the appropriate sulfonyl chloride (8.3 mmol) in pyridine for 2 h, filtered and then washed with NMP (3×), iPrOH (3×) and CH₂Cl₂ (5×).

Step 3

The resin (0.83 mmol) was agitated with 2M tin(II) chloride dihydrate in THF (6 mL) for 2 h, filtered and then washed with THF (3×), iPrOH (3×) and CH₂Cl₂ (5×).

Step 4

The resin (0.83 mmol) was agitated with iodobenzoic acid (8.3 mmol), PyBop (8.3 mmol) diisopropylethyl amine (16.6 mmol) in NMP overnight, filtered and then washed with NMP (3×), iPrOH (3×) and CH₂Cl₂ (5×).

Step 5

The resin (0.22 mmol) was agitated with the appropriate boronic acid (2.2 mmol), PdCl₂(dppf) CH₂Cl₂ (catalytic amount), tributyl phosphine (0.8 mmol) and triethyl amine (4.4 mmol) in anhydrous DMF at 70° C. for 2 days. The resin was then filtered and washed with NMP (3×), iPrOH (3×) and CH₂Cl₂ (5×).

Step 6

The product was cleaved from the resin with 1:1 trifluoroacetic acid/CH₂Cl₂ (30 min), filtered, concentrated in vacuo and purified via reverse phase HPLC (acetonitrile/water).

For compound 33 use steps 1,2 and 6.

For compound 35 use steps 1-3 and 6.

### Example 4

General Procedure for Synthesis of Compounds 21, 34

Step 1

Wang resin loaded with N-Fmoc-iodophenylalanine (4.6 mmol) was treated with 25% piperidine in DMF (2×10 min) and then washed with DMF (3×), iPrOH (3×) and CH₂Cl₂ (5×). The resin was then agitated with proline (28 mmol), diisopropyl carbodiimide (28 mmol) and HOBT (28 mmol) in NMP overnight. The resin was then filtered and washed with NMP (3×), iPrOH (3×) and CH₂Cl₂ (5×).

Step 2

The resin (4.6 mmol) was agitated with the appropriate sulfonyl chloride (46.2 mmol) in pyridine for 2 h, filtered and then washed with NMP (3×), iPrOH (3×) and CH₂Cl₂ (5×).

Step 3

The resin (0.22 mmol) was agitated with the appropriate boronic acid (2.2 mmol), PdCl₂(dppf) CH₂Cl₂ (catalytic amount), tributyl phosphine (0.8 mmol) and triethyl amine (4.4 mmol) in anhydrous DMF at 70° C. for 2 days. The resin was then filtered and washed with NMP (3×), iPrOH (3×) and CH₂Cl₂ (5×).

Step 4

The product was cleaved from the resin with 1:1 trifluoroacetic acid/CH₂Cl₂ (30 min), filtered, concentrated in vacuo and purified via reverse phase HPLC (acetonitrile/water).

### Example 5

General Procedure for Synthesis of Compound 29

Step 1

Wang resin loaded with N-Fmoc-nitrophenylalanine (0.35 mmol) was agitated with 2M tin(II) chloride hydrate in THF (10 mL) for 2 h, filtered and then washed with THF (3×), iPrOH (3×) and CH₂Cl₂ (5×).

Step 2

The resin (0.35 mmol) in CH₂Cl₂ was cooled to −78° C. and bromoacetic acid (0.72 mmol) was added. After 45 min diisopropyl carbodiimide (0.48 mmol) was added and the reaction was allowed to warm to RT overnight. The resin was filtered and washed with CH₂Cl₂ (5×).

Step 3

The resin (0.35 mmol) was treated with 25% piperidine in DMF (2×10 min) and then washed with DMF (3×), iPrOH (3×) and CH₂Cl₂ (5×).

Step 4

The resin (0.35 mmol) in CH₂Cl₂ was cooled to −78° C., and N-(methylsulfonyl)proline (3.5 mmol) was added. After 45 min diisopropyl carbodiimide (0.48 mmol) was added and the reaction was allowed to warm to RT overnight. The resin was filtered and washed with CH₂Cl₂ (5×).

Step 5

The product was cleaved from the resin with 1:1 trifluoroacetic acid/CH₂Cl₂ (30 min), filtered, concentrated in vacuo and purified via reverse phase HPLC (acetonitrile/water).

### Example 6

General Procedure for Synthesis of Compounds 5, 8, 26, 27, 28

Step 1

Triethyl amine (0.55 mol) was added to a solution of H-Pro-(OMe) HCl (0.21 mol) in CH₂Cl₂ (50 mL). The resulting slurry was filtered, the filtrate cooled to 0° C. and the appropriate sulfonyl chloride (0.2 mol) in CH₂Cl₂ was added dropwise. The reaction was allowed to warm to RT, stirred for 4 h, washed with water (1×), 5% citric acid solution (2×), 1N sodium hydroxide solution (3×) and saturated sodium chloride solution (1×), dried (MgSO₄) and concentrated in vacuo to give a brown oil which was recrystallized from hexanes/ethyl acetate.

Step 2

The methyl ester from step 1 was dissolved in 2N lithium hydroxide (120 mL). After 1 h the reaction was washed with ether and the aqueous phase acidified with 5% citric acid. The aqueous solution was extracted with CH₂Cl₂ and the combined organic phases were concentrated in vacuo to give a white solid.

Step 3

A solution of the carboxylic acid from step 2 (4.5 mmol), H-Tyr (But)-OMe (4.5 mmol), HATU (4.5 mmol) and diisopropylethyl amine (13.5 mmol) in DMF (25 mL) was stirred at RT for 3 h. The reaction was then diluted with ethyl acetate (100 mL), washed with 5% NaHCO₃ (25 mL), water (2×25 mL) and saturated sodium chloride solution (25 mL), dried (MgSO₄) and concentrated in vacuo to give product. The product was treated with trifluoroacetic acid/water (50 mL) for 1 h and then concentrated in vacuo.

Step 4

A slurry of the ether from step 3 (1.2 mmol) and anhydrous K₂CO₃ (24 mmol) in acetone (10 mL) was stirred a RT for 0.5 h. The appropriate benzyl chloride (5.8 mmol) was added and the reaction warmed to 50° C. for 24 h. The reaction was filtered and concentrated in vacuo.

Step 5

Lithium hydroxide (2.4 mmol) was added to a solution of the ester in 1/1 THF/water (5 mL). After 1 h the reaction was neutralized with 10% citric acid solution and extracted with ethyl acetate (20 mL). The organic phase was washed with saturated sodium chloride, dried (MgSO₄) and concentrated in vacuo. The crude product was purified via reverse phase HPLC (acetonitrile/water).

For compound 5 omit step 4

### Example 7

General Procedure for Synthesis of Compounds 25, 30

Step 1

Example 6, steps 1-3

Step 2

A slurry of the ether from step 1 (1.1 mmol), cesium carbonate (3.7 mmol) and 1-bromo-2 chloroethane (1.11 mmol) in THF was refluxed for 3 days. The reaction slurry was then filtered, concentrated in vacuo, diluted with ethyl acetate (25 mL), filtered, washed with aqueous sodium bicarbonate solution, dried (MgSO₄) and concentrated in vacuo.

Step 3

Sodium azide (0.79 mmol) and catalytic sodium iodide were added to a solution of the chloride from step 2 (0.72 mmol) in DMF (25 mL). The reaction was warmed to 70° C. for 16 h, diluted with ethyl acetate (200 mL), washed with water (2×50 mL), dried (MgSO₄) and concentrated in vacuo.

Step 4

A solution of the product from step 3, tin(II) chloride dihydrate (3.9 mmol), thiophenol (1.4 mmol) and triethyl amine (2.8 mmol) in THF was stirred at RT for 3 h. The reaction mixture was then concentrated in vacuo, diluted with ethyl acetate (50 mL) and extracted with 10% citric acid (3×10 mL). The combined aqueous phases were made basic with 1N sodium bicarbonate and extracted with ethyl acetate (2×25 mL). The combined organic phases were washed with water (2×10 mL) and saturated sodium chloride (10 mL), dried (MgSO₄) and concentrated in vacuo.

Step 5

The amine from step 4 was dissolved in 1:1 acetic anhydride/CH₂Cl₂, stirred for 0.75 h and concentrated in vacuo.

Step 6

Lithium hydroxide was added to ester in 1:1 THF/water at RT. After 2 h the reaction was neutralized with 10% citric acid, concentrated in vacuo and purified via reverse phase HPLC (acetonitrile/water).

For compound 25 omit step 5.

### Example 8

General Procedure for Synthesis of Compounds 10, 11, 12

Step 1

Example 6, steps 1-3

Step 2

A slurry of the ether from step 1 (0.1 mmol), the appropriate 1-(2-chloroethyl)amine (0.4 mmol), anhydrous potassium carbonate (1 mmol) and catalytic sodium iodide in acetone was warmed to reflux. The reaction was filtered and concentrated in vacuo when all starting material was consumed via HPLC analysis.

Step 3

Lithium hydroxide was added to ester in 1:1 THF/water at RT. After 2 h the reaction was neutralized with 10% citric acid, concentrated in vacuo and purified via reverse phase HPLC (acetonitrile/water).

### Example 9

General Procedure for Synthesis of Compounds 19, 23

Step 1

A slurry of potassium carbonate (100 mmol), 1-bromo-2-chloroethane (10 mmol), catalytic sodium iodide and Boc-Tyr-OMe (10 mmol) in acetone (50 mL) was warmed to reflux for 2 h. The reaction was allowed to cool to RT, filtered and concentrated in vacuo.

Step 2

The compound from step 1 (2 mmol) was treated with 1:1 trifluoracetic acid/CH₂Cl₂ for 0.5 h and concentrated in vacuo. The resulting oil was dissolved in DMF and a carboxylic acid (example 6 steps 1, 2; 2 mmol), HATU (2 mmol) and diisopropylethyl amine (6 mmol) were added. The reaction was stirred at RT for 2-3 h, concentrated in vacuo, diluted with ethyl acetate, washed with 5% sodium bicarbonate, water and saturated sodium chloride, dried (MgSO₄) and concentrated in vacuo to give desired product.

Step 3

A solution of the compound from step 2 (0.89 mmol), tert-butyl 1-piperazinecarboxylate (0.89 mmol) and diisopropylethyl amine (1.8 mmol) in THF was stirred at RT for 2-3 h. The reaction was then concentrated in vacuo, diluted with ethyl acetate, washed with water and saturated sodium chloride, dried (MgSO₄) and concentrated in vacuo to give desired product.

Step 4

The compound from step 3 (0.18 mmol) was treated with 1:1 trifluoracetic acid/CH₂Cl₂ for 0.5 h and concentrated in vacuo.

Step 5

The compound from step 4 was dissolved in CH₂Cl₂, excess triethylamine and acetic anhydride (0.18 mmol) were added. The reaction was concentrated in vacuo, diluted with ethyl acetate, washed with water and saturated sodium chloride, dried (MgSO₄), and concentrated in vacuo to give desired product.

Step 6

Lithium hydroxide was added to a solution of ester (0.18 mmol) in 1:1 THF/water. After 2 h the reaction was neutralized with 10% citric acid, concentrated in vacuo and purified via reverse phase HPLC (acetonitrile/water).

For compound 19 omit step 5

### Example 10

General Procedure for Synthesis of Compounds 6, 39, 40

Step 1

Wang resin loaded with N-Fmoc-p-nitrophenylalanine (0.68 mmol) was treated with 20% piperidine in DMF (15 min) and then washed with methanol (2×) and CH₂Cl₂ (2×). The resin was treated again with 20% piperidine in DMF (30 min) and then washed with methanol (4×) and CH₂Cl₂ (4×). The resin was then agitated with Fmoc-proline (2.2 mmol) and diisopropyl carbodiimide (2.0 mmol) in CH₂Cl₂ for 24 h. The resin was then filtered and washed with methanol (4×) and CH₂Cl₂ (4×).

Step 2

The resin (0.68 mmol) was treated with 20% piperidine in DMF (15 min) and then washed with methanol (2×) and CH₂Cl₂ (2×). The resin was treated again with 20% piperidine in DMF (30 min) and then washed with methanol (4×) and CH₂Cl₂ (4×). The resin then was agitated with the appropriate sulfonyl chloride (3.4 mmol) and pyridine (5 mL) in THF (5 mL) overnight, filtered and washed with THF (4×) and CH₂Cl₂ (4×).

Step 3

The resin (0.68 mmol) was agitated with 2M tin(II) chloride dihydrate in DMF (8.2 mL) for 24 h, filtered and then washed with saturated sodium bicarbonate (1×), methanol (4×) and CH₂Cl₂ (4×).

Step 4

The resin (0.25 mmol) was agitated with the appropriate carboxylic acid (1.9 mmol) and diisopropyl carbodiimide (1.3 mmol) in NMP (4 mL) overnight. The resin was then filtered and washed with methanol (4×) and CH₂Cl₂ (4×).

Step 5

The product was cleaved from the resin with 1:1 trifluoroacetic acid/CH₂Cl₂ (30 min), filtered, concentrated in vacuo and purified via reverse phase HPLC (acetonitrile/water).

### Example 11

General Procedure for Synthesis of Compound 7

Step 1

Example 10, steps 1-3

Step 2

The resin from step 1 (0.31 mmol) was agitated with the appropriate sulfonyl chloride (41 mmol) in 1:1 pyridine/NMP (4 mL) overnight, filtered and then washed with methanol (4×) and CH₂Cl₂ (4×).

Step 3

The product was cleaved from the resin with 1:1 trifluoroacetic acid/CH₂Cl₂ (30 min), filtered, concentrated in vacuo and purified via reverse phase HPLC (acetonitrile/water).

### Example 12

General Procedure for Synthesis of Compounds 2, 3, 9, 31, 36, 37

Step 1

A slurry of Wang resin (0.83 mmol) and the appropriate Fmoc protected aminoacid (2.5 mmol) in DMF (7 mL) was agitated for 15 min. Pyridine (4.2 mmol) and 2,6-dichlorobenzoylchloride (2.5 mmol) were then added and the slurry agitated for an additional 20 h. The resin was filtered and washed with DMF (3×), iPrOH (3×) and CH₂Cl₂ (5×).

Step 2

The resin (0.79 mmol) was treated with 25% piperidine in DMF (2×10 min) and then washed with DMF (3×), iPrOH (3×) and CH₂Cl₂ (5×). The resin was then agitated with Fmoc-Pro-OH (8 mmol), diisopropyl carbodiimide (8 mmol) and HOBT (8 mmol) in NMP overnight. The resin was then filtered and washed with NMP (3×), iPrOH (3×) and CH₂Cl₂ (5×).

Step 3

The resin (0.79 mmol) was treated with 25% piperidine in DMF (2×10 min) and then washed with DMF (3×), iPrOH (3×) and CH₂Cl₂ (5×). The resin was agitated with the appropriate sulfonyl chloride (8 mmol) and pyridine (8 mmol) in DMF for 2 h, filtered and then washed with NMP (3×), iPrOH (3×) and CH₂Cl₂ (5×).

Step 4

The product was cleaved from the resin with 1:1 trifluoroacetic acid/CH₂Cl₂ (30 min), filtered, concentrated in vacuo and purified via reverse phase HPLC (acetonitrile/water).

### Example 13

General Procedure for Synthesis of Compound 24

Step 1

Example 10, steps 1-3

Step 2

A solution of Fmoc-(L)-Lys(Boc)-OH (3.5 mmol), HOBt (3.5 mmol) and PyBop (3.3 mmol) in anhydrous DMF was added to resin from step 1 (0.34 mmol). Diisopropylamine (6.8 mmol) was added and the slurry agitated for 4 days at RT. The resin was filtered and washed with methanol (4×) and CH₂Cl₂ (4×).

Step 3

p-Nitrophenyl chloroformate (3.5 mmol) and diisopropylethyl amine (3.4 mmol) were added to a slurry of resin (0.34 mmol) in 1:1 THF/CH₂Cl₂ (6 mL). The mixture was agitated for 1 h, filtered and washed with THF (4×) and CH₂Cl₂. DBU (3.4 mmol) was added to the resin in anhydrous DMF (5 mL) and the slurry was agitated for 24 h. The resin was then filtered and washed with DMF (3×), THF (4×) and CH₂Cl₂ (4×).

Step 4

The resin from step 3 was agitated in 95% trifluoroacetic acid/water for 1 h, filtered and concentrated in vacuo. CH₂Cl₂ (10 mL), acetic anhydride (6.4 mmol) and diisopropylethyl amine (4.6 mmol) were added to the residue and the resulting solution stirred at RT for 1 h. The solvent was removed in vacuo, the residue dissolved in 1N hydrochloric acid (40 mL) and extracted with ethyl acetate (3×15 mL). The combined organic phases were washed with saturated sodium chloride (10 mL), dried (MgSO₄), concentrated in vacuo and purified via reverse phase HPLC (acetonitrile/water).

### Example 14

General Procedure for Synthesis of Compounds 20, 22, 41

Step 1

A solution of H-Hyp-OMe.HCl (3.43 mmol) and the appropriate sulfonyl chloride (10.5 mmol) in 1:1 pyridine/CH₂Cl₂ (20 mL) was stirred at RT overnight. The reaction was acidified with 1N hydrochloric acid and extracted with CH₂Cl₂. The combined organic phases were dried (MgSO₄) and concentrated in vacuo to give desired product.

Step 2

A solution of the compound from step 1 (3.0 mmol) and sodium azide (14.9 mmol) in DMF (15 mL) was warmed to 70° C. overnight. The reaction was cooled to RT, quenched with saturated sodium bicarbonate solution and extracted with ethyl acetate. The combined organic phases were dried (MgSO₄) and concentrated in vacuo to give the desired product.

Step 3

Excess lithium hydroxide was added to a solution of the compound from step 2 in 1:1 THF/water (200 mL). The reaction was stirred at RT for 3 h, the volatiles removed in vacuo and the remaining aqueous solution acidified with 1N hydrochloric acid. The aqueous solution was extracted with ethyl acetate, the combined organic phases dried (MgSO₄) and concentrated in vacuo to give the desired product.

Step 4

A solution of anhydrous potasium cabonate (34 mmol), BocTyr-OMe (1.0 mmol), 1-(2-chloroethyl) piperidine (3.8 mmol) and catalytic sodium iodide in acetone (100 mL) was warmed to reflux for 6.5 h. The reaction was cooled to RT, filtered and concentrated in vacuo. The resulting oil was dissolved in 1:1 methylene chloride/triflouroacetic acid (8 mL), stirred at RT for 2 h and concentrated in vacuo to give amine.

Step 5

A solution of the compound from step 3 (0.75 mmol), the compound from step 4 (0.75 mmol), HATU (0.74 mmol) and diisopropylethyl amine (1.46 mmol) in DMF (10 mL) was stirred at RT for 1.5 h. The volatiles were then removed in vacuo, the residue dissolved in 5% sodium bicarbonate and extracted with ethyl acetate. The combined organic phases were washed with saturated sodium chloride, dried (MgSO₄) and concentrated in vacuo.

Step 6

A solution of the product from step 5, tin(II) chloride dihydrate (0.72 mmol), thiophenol (2.9 mmol) and triethyl amine (3.6 mmol) in 1:1 DMF/THF was stirred at RT for 3 h. The reaction mixture was then concentrated in vacuo, partioned between sodium carbonate and ethyl acetate. The organic phase was washed with water and saturated sodium chloride, dried (MgSO₄) and concentrated in vacuo.

Step 7

The compound from step 6 was dissolved in CH₂Cl₂, excess triethylamine and acetic anhydride were added. The reaction was concentrated in vacuo, diluted with ethyl acetate, washed with water and saturated sodium chloride, dried (MgSO₄), and concentrated in vacuo to give desired product.

Step 8

Excess lithium hydroxide was added to a solution of the compound from the previous step in 1:1 THF/water. The reaction was stirred at RT for 3 h, the volatiles removed in vacuo and the remaining aqueous solution acidified with 1N hydrochloric acid. The aqueous solution was extracted with ethyl acetate, the combined organic phases dried (MgSO₄), concentrated in vacuo, and purified via reverse phase HPLC (acetonitrile/water) to give the desired product.

For compounds 20, 41 omit step 7

### Example 15

General Procedure for Synthesis of Compound 44

Step 1

Example 14, steps 1-3

Step 2

A solution of the carboxylic acid from step 1, H-Tyr(But)-OMe, HATU and diisopropylethyl amine in DMF was stirred at RT for 3 h. The reaction was then diluted with ethyl acetate, washed with 5% NaHCO₃, water and saturated sodium chloride solution, dried (MgSO₄) and concentrated in vacuo to give product.

Step 3

A solution of the product from step 2, tin(II) chloride dihydrate (1 eq), thiophenol (4 eq) and triethyl amine (5 eq) in 1:1 DMF/THF was stirred at RT for 3 h. The reaction mixture was then concentrated in vacuo, partioned between sodium carbonate and ethyl acetate. The organic phase was washed with water and saturated sodium chloride, dried (MgSO₄) and concentrated in vacuo.

Step 4

Excess lithium hydroxide was added to a solution of the compound from the previous step in 1:1 THF/water. The reaction was stirred at RT for 3 h, the volatiles removed in vacuo and the remaining aqueous solution acidified with 1N hydrochloric acid. The aqueous solution was extracted with ethyl acetate, the combined organic phases dried (MgSO₄) and concentrated in vacuo.

Step 5

The compound from the previous step was treated with trifluoroacetic acid (30 min), concentrated in vacuo and purified via reverse phase HPLC (acetonitrile/water).

### Example 16

General Procedure for Synthesis of Compound 42

Step 1

A solution of H-Hyp-OMe.HCl (3.43 mmol) and the appropriate sulfonyl chloride (10.5 mmol) in 1:1 pyridine/CH₂Cl₂ (20 mL) was stirred at RT overnight. The reaction was acidified with 1N hydrochloric acid and extracted with CH₂Cl₂. The combined organic phases were dried (MgSO₄) and concentrated in vacuo to give desired product.

Step 2

A solution of the compound from Example 14, step 4, the compound from step 1, HATU and diisopropylethyl in DMF were stirred at RT for 1.5 h. The volatiles were then removed in vacuo, the residue dissolved in 5% sodium bicarbonate and extracted with ethyl acetate. The combined organic phases were washed with saturated sodium chloride, dried (MgSO₄) and concentrated in vacuo.

Step 3

Excess lithium hydroxide was added to a solution of the compound from the previous step in 1:1 TMF/water. The reaction was stirred at RT for 3 h, the volatiles removed in vacuo and the remaining aqueous solution acidified with 1N hydrochloric acid. The aqueous solution was extracted with ethyl acetate, the combined organic phases dried (MgSO₄), concentrated in vacuo, and purified via reverse phase HPLC (acetonitrile/water) to give the desired product.

### Example 17

General Procedure for Synthesis of Compound 43

Step 1

A solution of H-Pro-(OMe) HCl (2.3 mmol) and the appropriate nitro substituted sulfonyl chloride (4.4 mmol) in pyridine (5 mL) was stirred at RT for 15 min and then partioned between ethyl acetate and 1N hydrochloric acid solution. The organic phase was dried (MgSO₄) and concentrated in vacuo.

Step 2

The methyl ester in step 1 was dissolved in 1:1 THF/2N lithium hydroxide solution and stirred overnight. The volatiles were removed in vacuo and the aqueous phase acidified with 1N hydrochloric acid. The aqueous solution was extracted with ethyl acetate and the combined organic phases were concentrated in vacuo to give a white solid.

Step 3

A solution of the compound from Example 14, step 4 (0.26 mmol), the carboxylic acid from step 2 (0.27 mmol), HATU (0.48 mmol) and diisopropylethyl amine (0.52 mmol) in DMF (5 mL) was stirred at RT overnight. The reaction was then partioned between ethyl acetate and 5% sodium bicarbonate. The organic phase was washed with saturated sodium chloride, dried (MgSO₄) and concentrated in vacuo.

Step 4

Raney nickel was washed with water (4×) and ethanol (3×) until the decanted solution was clear. The Raney nickel was covered with methanol and the compound from step 3 in methanol was added. After 2.5 h the slurry was filtered through a celite plug and concentrated in vacuo.

Step 5

A solution of the compound from step 4 (0.98 mmol), Boc-Gly-OH (0.99 mmol), HATU (0.98 mmol) and diisopropylethyl amine (1.98 mmol) in DMF (5 mL) was stirred at RT for 1.5 h. The reaction was then partioned between ethyl acetate and 5% sodium bicarbonate. The organic phase was washed with saturated sodium chloride, dried (MgSO₄) and concentrated in vacuo.

Step 6

Excess lithium hydroxide was added to a solution of the compound from the previous step in 1:1 THF/water. The reaction was stirred at RT for 3 h, the volatiles removed in vacuo and the remaining aqueous solution acidified with 1N hydrochloric acid. The aqueous solution was extracted with ethyl acetate, the combined organic phases dried (MgSO₄), concentrated in vacuo, and purified via reverse phase HPLC (acetonitrile/water) to give the desired product.

### Example 18

General Procedure for Synthesis of Compound 32

Step 1

Example 6, steps 1-3

Step 2

A solution of the compound from step 1, the appropriate aminocarbonyl chloride (1.1 eq) and triethylamine (2.2 eq) in methylene chloride was stirred at 0° C. overnight. The reaction was diluted with ethyl acetate, washed with 5% citric acid, 5% sodium bicarbonate and saturated sodium chloride, dried (MgSO₄) and concentrated in vacuo.

Step 3

Excess lithium hydroxide was added to a solution of the compound from the previous step in 1:1 THF/water. The reaction was stirred at RT for 3 h, the volatiles removed in vacuo and the remaining aqueous solution acidified with 1N hydrochloric acid. The aqueous solution was extracted with ethyl acetate, the combined organic phases dried (MgSO₄), concentrated in vacuo and purified via reverse phase HPLC (acetonitrile/water) to give the desired product.

1. A method for inhibiting activity of a plurality of integrins containing a φ1 subunit, the method comprising administering to a system containing said integrins, an effective amount of a pan-β1 integrin antagonist compound of Formula 1: 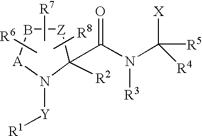
or a pharmaceutically acceptable salt thereof wherein:
R¹ is
1) C1-10 alkyl,
2) C2-10alkenyl,
3) C2-10 alkynyl,
4) Cy,
5) Cy—C1-10 alkyl,
6) Cy—C2-10 alkenyl,
7) Cy—C2-C10 alkynyl,
wherein alkyl, alkenyl, and alkynyl are optionally substituted with one to four substituents independently selected from R^{a}; and Cy is optionally substituted with one to four substituents independently selected from R^{b};
R² is
1) hydrogen,
2) C1-10 alkyl,
3) C2-10 alkenyl,
4) C2-10 alkynyl,
5) aryl,
6) aryl-C1-10 alkyl,
7) heteroaryl,
8) heteroaryl-C1-10 alkyl,
wherein alkyl, alkenyl, and alkynyl are optionally substituted with one to four substituents independently selected from R^{a} and aryl and heteroaryl are optionally substituted with one to four substituents independently selected from R^{b};
R³ is
1) hydrogen,
2) C1-10 alkyl,
3) Cy, or
4) Cy—C1-10 alkyl,
wherein alkyl is optionally substituted with one to four substituents independently selected from R^{a}; and Cy is optionally substituted with one to four substituents independently selected from R^{b};
R⁴ is
1) hydrogen,
2) C1-10 alkyl,
3) C2-10 alkenyl,
4) C2-10 alkynyl,
5) Cy,
6) Cy—C1-10 alkyl,
7) Cy—C2-10 alkenyl,
8) Cy—C2-10 alkynyl,
wherein alkyl, alkenyl and alkynyl are optionally substituted with one to four substituents selected from phenyl and R^{x}, and Cy is optionally substituted with one to four substituents independently selected from R^{y}; or
R³, R⁴ or R³, R⁵ and the atoms to which they are attached together form a mono- or bicyclic ring containing 0-2 additional heteroatoms selected from N, O, and S;
R⁵ is
1) hydrogen,
2) C1-10 alkyl,
3) C2-10 alkenyl,
4) C2-10 alkynyl,
5) aryl,
6) aryl-C1-10 alkyl,
7) heteroaryl,
8) heteroaryl-C1-10 alkyl,
wherein alkyl, alkenyl and alkynyl are optionally substituted with one to four substituents selected from R^{x} and aryl and heteroaryl are optionally substituted with one to four substituents independently selected from R^{y}; or
R⁴, R⁵ and the carbon to which they are attached form a 3-7 membered mono- or bicyclic ring containing 0-2 heteroatoms selected from N, O and S;
R⁶, R⁷, and R⁸ are each independently selected from the group consisting of
1) a group selected from R^{d}, and
2) a group selected from R^{x}; or
two of R⁶, R⁷, and R⁸ and the atom to which both are attached, or two of R⁶, R⁷, and R⁸ and the two adjacent atoms to which they are attached, together form a 5-7 membered saturated or unsaturated monocyclic ring containing zero to three heteroatoms selected from N, O or S,
R¹² is
1) hydrogen,
2) C1-10 alkyl,
3) C2-10 alkenyl,
4) C2-10 alkynyl,
5) Cy,
6) Cy—C1-10 alkyl,
7) Cy—C2-10 alkenyl,
8) Cy—C2-10 alkynyl,
wherein alkyl, alkenyl and alkynyl are optionally substituted with one to four substituents selected from phenyl and R^{x}, and Cy is optionally substituted with one to four substituents independently selected from R^{y};
R¹³ is
1) hydrogen,
2) C1-10 alkyl,
3) C2-10 alkenyl,
4) C2-10 alkynyl,
5) aryl,
6) aryl-C1-10 alkyl,
7) heteroaryl,
8) heteroaryl-C1-10 alkyl,
wherein alkyl, alkenyl and alkynyl are optionally substituted with one to four substituents selected from R^{x} and aryl and heteroaryl are optionally substituted with one to four substituents independently selected from R^{y};
R^{a} is
1) Cy, or
2) a group selected from R^{x};
wherein Cy is optionally subsituted with one to four substituents independently selected from R^{c};
R^{b} is
1) a group selected from R^{a},
2) C1-10 alkyl,
3) C2-10 alkenyl,
4) C2-10 alkynyl,
5) aryl C1-10 alkyl,
6) heteroaryl C1-10 alkyl,
wherein alkyl, alkenyl, alkynyl, aryl, heteroaryl are optionally substituted with a group independently selected from R^{c};
R^{c} is
1) halogen,
2) NO₂,
3) C(O)OR^{f}
4) C1-4 alkyl,
5) C1-4 alkoxy,
6) aryl,
7) aryl C1-4 alkyl,
8) aryloxy,
9) heteroaryl,
10) NR^{f}R^{g},
11) NR^{f}C(O)R^{g},
12 NR^{f}C(O)NR^{f}R^{g},
13) CN;
14) C(O)Cy; or
15) C(O)alkyl.
R^{d} and R^{e} are independently selected from hydrogen, C1-10 alkyl, C2-10 alkenyl, C2-10 alkynyl, Cy and Cy C1-10 alkyl, aryl, heteroaryl, aryl-substituted aryl, aryl substituted heteroaryl, heteroaryl-substituted heteroaryl
wherein alkyl, alkenyl, alkynyl, heteroaryl and Cy is optionally substituted with one to four substituents independently selected from R^{c};
R^{d} and R^{e} together with the atoms to which they are attached form a heterocyclic ring of 5 to 7 members containing 0-2 additional heteroatoms independently selected from oxygen, sulfur and nitrogen,
R^{f} and R^{g} are independently selected from hydrogen, C1-10 alkyl, Cy and Cy—C1-10 alkyl wherein Cy is optionally substituted with C1-10 alkyl; or R^{f} and R^{g} together with the carbon to which they are attached form a ring of 5 to 7 members containing 0-2 heteroatoms independently selected from oxygen, sulfur and nitrogen, wherein the nitrogen is optionally substituted with C(O)R^{e}, SO₂R^{e}; or SO₂NR^{d}R^{e};
R^{h} is
1) hydrogen,
2) C1-10 alkyl,
3) C2-10 alkenyl,
4) C2-10 alkynyl,
5) cyano,
6) aryl,
7) aryl C1-10 alkyl,
8) heteroaryl,
9) heteroaryl C1-10 alkyl, or
10) —SO2Rⁱ;
wherein alkyl, alkenyl, and alkynyl are optionally substituted with one to four substituents independently selected from R^{a}; and aryl and heteroaryl are each optionally substituted with one to four substituents independently selected from R^{b};
Rⁱ is
1) C1-10 alkyl,
2) C2-10 alkenyl,
3) C2-10 alkynyl, or
4) aryl;
wherein alkyl, alkenyl, alkynyl and aryl are each optionally substituted with one to four substituents independently selected from R^{c};
R^{j} is selected from hydrogen, C1-10 alkyl, C2-10 alkenyl, C2-10 alkynyl, Cy and Cy C1-10 alkyl, aryl, heteroaryl, aryl-substituted aryl, aryl substituted heteroaryl, heteroaryl-substituted heteroaryl
wherein alkyl, alkenyl, alkynyl, heteroaryl, and Cy is optionally substituted with one to four substituents independently selected from R^{c}
R^{x} is
1) —OR^{d},
2) —NO₂,
3) halogen,
4) —S(O)ₘR^{d}
5) —SR^{d},
6) —S(O)₂OR^{d},
7) —S(O)ₘNR^{d}R^{e},
8) —NR^{d}R^{e},
9) —O(CR^{f}R^{g})ₙNR^{d}R^{e},
10) —C(O)R^{d},
11) —CO₂R^{d},
12) —CO₂(CR^{f}R^{g})ₙCONR^{d}R^{e},
13) —OC(O)R^{d},
14) —CN,
15) —C(O)NR^{d}R^{e},
16) —NR^{d}C(O)R^{e},
17) —OC(O)NR^{d}R^{e},
18) —NR^{d}C(O)OR^{e},
19) —NR^{d}C(O)NR^{d}R^{e},
20) —CR^{d}(N—OR^{e}),
21) —CF₃,
22) oxo,
23) NR^{d}C(O)NR^{d}SO₂Rⁱ,
24) NR^{d}S(O)ₘR^{e},
25) —OS(O)₂OR^{d},
26) —OP(O)(OR^{d})₂; or 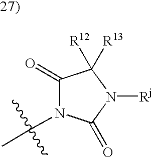
R^{y} is
1) a group selected from R^{x},
2) C1-10 alkyl,
3) C2-10 alkenyl,
4) C2-10 alkynyl,
5) aryl C1-10 alkyl,
6) heteroaryl C1-10 alkyl,
7) cycloalkyl,
8) heterocyclyl;
9) aryl;
10) heteroaryl
wherein alkyl, alkenyl, alkynyl, heteroaryl and aryl are each optionally substituted with one to four substituents independently selected from R^{x};
Cy is
cycloalkyl, heterocyclyl, aryl, or heteroaryl;
m is an integer from 1 to 2;
n is an integer from 1 to 10;
X is
1) —C(O)OR^{d}
2) -5-tetrazolyl;
3) —(CR^{f}R^{g})COOR^{d}
Y is
1) —S(O)₂—; or
2) —S(O)₂NR^{e}
Z and A are independently selected from —C— and —C—C—;
B is selected from the group consisting of
1) a bond,
2) —C—,
3) —C—C—;
4) —C═C—,
5) a heteroatom selected from the group consisting of nitrogen, oxygen, and sulfur; and
5) —S(O)ₘ—.

2. A method of claim 1, which comprises administering an effective amount of a compound of Formula 2: 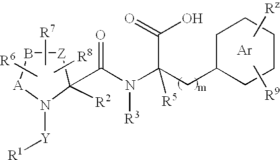
Wherein Ar is aryl, heteroaryl, aryl-substituted aryl, aryl substituted heteroaryl, heteroaryl-substituted heteroaryl
and R^{z} is:
a) —OR^{d}
b) —NH₂
c) NHR^{d}
d) —NR^{d}C(O)R^{e}
e) —NR^{d}S(O)ₘR^{e}; and
wherein R^{d} and R^{e} are as recited in claim 1 and R⁹ is selected from H and R^{y}.

3. A method of claim 2, which comprises administering an effective amount of a compound of Formula 3: 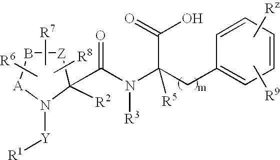
wherein Rz is as recited in claim 2 and R⁹ is selected from H and R^{y}.

4. A method for inhibiting a plurality of integrins containing a β1 subunit, wherein the method comprises administering to a system containing said integrins an effective amount of a compound of claim 2 or a pharmaceutically acceptable salt thereof, wherein Ar is aryl, heteroaryl, aryl-substituted aryl, aryl substituted heteroaryl, heteroaryl-substituted heteroaryl
and R^{z} is:
a) —OR^{d}
b) —NH₂
c) NHR^{d}
d) —NR^{d}C(O)R^{e}
e) —NR^{d}S(O)ₘR^{e}; wherein R^{d} and R^{e} are as recited in claim 1 and and R⁹ is selected from H and R^{y}.

5. A method for inhibiting a plurality of integrins containing a β1 subunit, the method comprisings administering to a system containing said integrins an effective amount of a compound of claim 3 or a pharmaceutically acceptable salt thereof, wherein R^{z} is as recited in claim 2.

6. A method of claim 3, which comprises administering an effective amount of a compound of Formula 4: 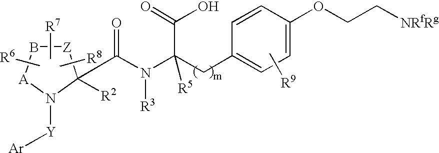
wherein Ar is aryl, heteroaryl, aryl-substituted aryl, aryl substituted heteroaryl which are optionally substituted with one to four substituents independently selected from R^{x} and R⁹ is selected from H and R^{y}.

7. A method of claim 1, which comprises administering an effective amount of a compound of Formula 5: 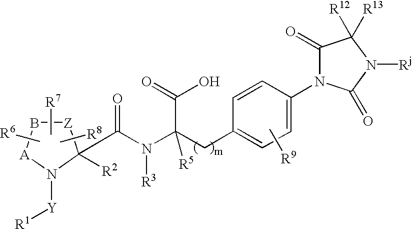
R⁹ is selected from H and R^{y}.

8. A method of claim 7, which comprises administering an effective amount of a compound of Formula 6: 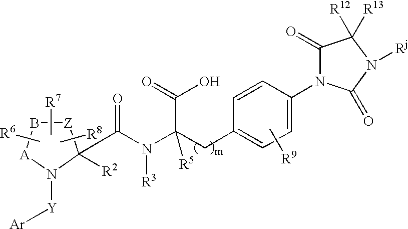
where Ar is aryl, heteroaryl, aryl-substituted aryl, aryl substituted heteroaryl which are optionally substituted with one to four substituents independently selected from R^{x} and R⁹ is selected from H and R^{y}.

9. A composition having the formula
D-L-P wherein:
D is a small organic molecule, said organic molecule attached to linker L, wherein linker L is not significantly cleavable using human enzymes, and wherein P is a biocompatible polymer.

10. The composition of claim 9 wherein,
D is a pan-beta1 integrin antagonist of Formula 1 having at least one bond selected from the group consisting of an amide, amine, ether, urea, thiourea, sulfonyl urea, sulfonyl amide, thio ether and carbon-carbon bond, and
L is 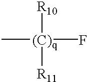
wherein R₁₀ and R₁₁ are independently selected from the group consisting of H, C₁₋₆ alkyls, aryls, substituted aryls, aralkyls, heteroalkyls, substituted heteroalkyls and substituted C₁₋₆ alkyls, q a positive integer and F is selected from O, NR¹, S, SO, SO₂, and wherein P is a polyalkylene oxide.

11. A method of inhibiting the activity of a plurality of β1-subunit containing integrins, comprising administering to a system containing said plurality, a pan-β1 antagonist selected from the group consisting of the small molecules of Table 1.
